SEQUENCE LISTING (1 sequence)

SEQ ID NO: 1 - protein, 8 aa, synthetic construct
  SITE 6: note = paranitrophenylalanine
KPAEFXRL

(19) United States
(12) Patent Application Publication
(10) Pub. No.: US 2024/0041764 A1
(43) Pub. Date: Feb. 8, 2024

(54) Sustained-Release Oral Fosamprenavir Formulation for Treatment of Reflux

(71) Applicants: The Medical College of Wisconsin, Inc., Milwaukee, WI (US); N-Zyme Biomedical Inc., Vancouver (CA)

(72) Inventors: Nikki Johnston, Oconomowoc, WI (US); Franco Gennaro Vigile, Nepean (CA)

(21) Appl. No.: 18/361,201

(22) Filed: Jul. 28, 2023

Related U.S. Application Data
(60) Provisional application No. 63/392,929, filed on Jul. 28, 2022.

(51) Int. Cl. A61K 9/00; A61K 31/635; A61K 31/665; A61K 47/36; A61P 1/04

(52) U.S. Cl. CPC A61K 9/0095; A61K 31/635; A61K 31/665; A61K 9/0053; A61K 47/36; A61P 1/04

(57) ABSTRACT

The present invention provides methods of treating airway reflux using an HIV protease inhibitor that is capable of binding to and inhibiting the enzymatic activity of pepsin. Compositions comprising sustained release formulations of HIV protease inhibitors are also provided for oral administration.

### CROSS-REFERENCE TO RELATED APPLICATIONS

This application claims priority to U.S. Application No. 63/392,929, filed on Jul. 28, 2022, the entire contents of which are hereby incorporated by reference.

### STATEMENT REGARDING FEDERALLY SPONSORED RESEARCH

N/A

### SEQUENCE LISTING

A Sequence Listing accompanies this application and is submitted as an XML file of the sequence listing named “650053_00979_Sequence_Listing” which is 2,375 bytes in size and was created on Jul. 25, 2023. The sequence listing is electronically submitted via Patent Center with the application and is incorporated herein by reference in its entirety.

### INTRODUCTION

Laryngopharyngeal reflux (LPR), the backflow of gastric contents into the laryngopharynx, is an important health problem. LPR affects children and adults equally, and the clinical spectrum of this disease is extensive.

More than 20% of the US population suffers from laryngopharyngeal reflux. While dietary/lifestyle modifications and alginates provide benefit to some, there is no gold standard medical therapy. Increasing evidence suggests that pepsin is partly, if not wholly, responsible for damage and inflammation caused by laryngopharyngeal reflux. A treatment specifically targeting pepsin would be amenable to local, inhaled delivery and could prove effective for endoscopic signs and symptoms associated with nonacid reflux.

Laryngopharyngeal reflux (LPR), the backflow of gastric contents into the laryngopharynx, is an important health problem. LPR affects both children and adults, and the clinical spectrum is extensive. Unlike patients with gastroesophageal reflux (GER) which is limited to the esophagus, many LPR patients do not experience acid indigestion but present with symptoms due to chronic laryngeal irritation, such as chronic cough, throat-clearing, post-nasal drip, dysphonia, globus, dysphagia, and dyspnea. Significant evidence supports the contribution of chronic LPR to serious and life-threatening illness including airway stenosis, reactive airway disease, and laryngeal cancer. LPR is estimated to affect more than 20% of the United States population and contribute to 10% visits to otolaryngologists. The economic burden of LPR is over $52 billion per year, which is 5.6-fold greater than that of GER; 52% of the burden is attributed to proton pump inhibitors (PPIs).

While PPI therapy is a mainstay in the treatment of GER disease (GERD), its efficacy for LPR is poor. In clinical practice, it was believed that patients with reflux laryngitis require higher doses and longer trials of PPIs than those with typical GERD given the assumption that the upper airway is more sensitive to acid reflux than the esophagus. However, placebo-controlled trials have failed to demonstrate therapeutic benefit of PPIs. While Reichel et al. and Lam et al. reported symptom improvement in randomized, double-blind, placebo-controlled trials, Vaezi argued that improvement was for heartburn rather than throat symptoms. Where laryngeal symptom improvement has been reported it was found proportionally higher in GERD patients than in those without GERD. Given the paucity of data supporting acid-suppression therapy for extraesophageal symptoms, the American Gastroenterological Association guidelines for GERD recommend against its use for acute treatment of patients with potential extraesophageal reflux (EER) syndromes (laryngitis, chronic cough) absent typical GERD symptoms. Despite such advice, treatment for LPR frequently continues to involve empiric therapy with PPIs.

While the acidity of reflux alone can damage the upper airways, combined multichannel intraluminal impedance-pH (MII-pH) monitoring has demonstrated that many episodes of LPR are nonacidic, and that weakly and nonacidic reflux is associated with persistent symptoms in acid-suppressed patients. These symptoms are alleviated by anti-reflux surgery and may be ameliorated by less invasive strategies that limit reflux occurrence or neutralize reflux constituents beyond acid (e.g. dietary and lifestyle modification and over-the-counter alginate products). Thus, one or more nonacid components of gastric refluxate must have a role in laryngeal damage. There is increasing evidence that pepsin, which is present in all refluxate, is partly, if not wholly, responsible for damage and inflammation caused by LPR.

Pepsin is a proteolytic enzyme which is synthesized and secreted as the zymogen pepsinogen by chief cells in the gastric fundus and subsequently cleaved upon introduction to the acidic stomach lumen to produce pepsin. Pepsin is maximally active at pH2 and retains activity up to pH6.5. While stable at pH8, pepsin is irreversibly inactivated at higher pH. The stomach and esophagus have intrinsic defenses against pepsin (mucus, peristalsis, and bicarbonate secretion), however laryngeal tissues do not. Pepsin is thought to play a key role in mucosal damage and inflammation during nonacidic reflux. At neutral pH, pepsin is taken up by laryngeal and hypopharyngeal cells by receptor-mediated endocytosis and retained in intracellular vesicles of low pH where it is presumed to be reactivated. The consequence is chronic inflammation, which in turn, gives rise to symptoms. Endocytosed nonacidic pepsin induces a proinflammatory cytokine gene expression profile in hypopharyngeal cells similar to that which contributes to disease severity during GERD. Inhibition of the proteolytic activity of pepsin abrogates this damage and inflammation.

With compelling evidence of nonacid proximal reflux of pepsin and its association with laryngeal and pharyngeal symptoms and endoscopic findings, the significant cost and risk of prolonged PPI therapy which continues to date despite its inefficacy in the absence of a gold standard medical therapy, and the limitations of alternative non-surgical treatment options such as the short-lived activity of over-the-counter products intended to provide temporary relief and the burden of adherence to dietary and lifestyle modifications, a new medical treatment which specifically targets pepsin would be of great benefit.

We and others have discussed the promise of inhibitors of peptic activity and/or receptor antagonists as potential new therapeutics for LPR.

### SUMMARY

In one aspect, the disclosure provides an oral sustained release formulation for treating reflux comprising: an effective amount of an HIV protease inhibitor; sodium alginate and a pharmaceutically acceptable carrier.

In another aspect, the disclosure provides a method of treating reflux in a subject in need thereof, the method comprising oral administering the formulation described herein to a subject to treat the reflux.

### BRIEF DESCRIPTION OF THE DRAWINGS

FIG. 1 shows a schematic of the assays used to screen for compounds that inhibit pepsin. Assay 1 (top) is a binding assay that measures how well a compound competes with fluorescently labeled pepstatin for its binding site on pepsin. Assay 2 (bottom) is a peptic activity assay that utilizes fluorescently labeled casein as an enzymatic substrate.

FIG. 2 shows the percent inhibition of pepsin produced by a library of pharmacologically active compounds that was screened using the binding assay.

FIGS. 3A-3B shows the co-crystallization structure of amprenavir bound to pepsin, with FIG. 3A showing 2|Fo|-|Fc| electron density map (green mesh) for amprenavir (purple carbons) bound in the pepsin active site (yellow carbons), and FIG. 3B showing a schematic view of the active site with amprenavir bound showing potential hydrogen bonding interactions as green, dashed lines.

FIGS. 3C and 3D shows the crystal structure and enzyme-inhibitor interactions in the active site of pepsin with darunavir bound.

FIGS. 4A-4D. 4 depict oral administration of Lexiva prevents pepsin-mediated airway epithelial damage in vivo. Representative animals from the different treatment protocols shown in each panel: pH 7 (FIG. 4A), Lexiva (FIG. 4B), 0.3 mg/ml pepsin at pH 7 (FIG. 4C), and 0.3 mg/ml pepsin pH 7+Lexiva (FIG. 4D). 20× magnification. (FIGS. 4A, 4B, and 4D) Normal-appearing respiratory epithelium consisting of a single layer of ciliated columnar epithelium with basal polarization of the nuclei and ciliated apical surfaces. (FIG. 4C) Reactive, multilayered epithelium with increased nuclear to cytoplasmic (N:C) ratio and cilia loss.

FIGS. 5A and 5B are a schematic of a 12-week, randomized, double blind, placebo-controlled clinical trial designed to test the efficacy of the HIV protease inhibitor Lexiva for the treatment of LPR.

FIG. 6 is the treatment schema of in vivo mouse study.

FIGS. 7A and 7B shows binding (FIG. 7A) and activity (FIG. 7B) curves of pepsin with HIV protease inhibitors.

FIGS. 8A-8D show pepsin and HIV protease inhibitor structural data. Left panels of FIGS. 8A-8D show the active site of porcine pepsin with HIV protease inhibitor bound. The 2Fo-Fc electron density map contoured at 1.0σ is shown as magenta mesh and the 2Fo-Fc simulated annealing composite omit map, also contoured at 1.0σ, is shown as green mesh. Right panels of FIGS. 8A-8D depict schematic views of the active site with HIV protease inhibitor bound showing potential hydrogen bonding interactions as green, dashed lines. Electron density maps were generated via POVSCRIPT and POV-Ray and schematic representation by MarvinSketch, and Adobe Illustrator.

FIGS. 9A-9H demonstrates laryngeal epithelial damage by pepsin and acid in vivo. Representative specimens from treatment groups. Paired images at 40× (FIGS. 9A-9D) and 200× (FIGS. 9E-9H) magnification collected rostral to vocal folds, representing larynx: pH7 (FIGS. 9A and 9E), pH4 (FIGS. 9B and 9F), 0.3 mg/ml pepsin at pH7 (FIGS. 9C and 9G), and 0.3 mg/ml pepsin at pH4 (FIGS. 9D and 9H). (FIGS. 9A and 9E) Normal respiratory columnar epithelium (arrow) about one cell layer thick with basal polarization of the nuclei and ciliated apical surfaces. (FIGS. 9B and 9F) Reactive epithelium characterized by thickening (fat arrow) and focal squamous epithelia (long arrow) with loss of cilia. In other areas, relative thickening of the mucosa with moderately increased nuclear to cytoplasmic (N:C) ratio and irregular, condensed chromatin is seen. (c,g) Thickened respiratory epithelium with pseudostratification of the epithelial cells. Keratinization (arrow) is present in multiple foci. Significant increase in the N:C ratio with loss of nuclear polarization and reduction in the apical cilia is evident in several regions of this treatment group. (d,h) Respiratory epithelium is necrotic (arrow) and replaced by an inflammatory exudate. A brisk, acute inflammatory infiltrate infiltrates the submucosal area. Scale bars FIGS. 9A-9D=100 μm; FIGS. 9E-9H=50 μm.

FIGS. 10A and 10B shows fosamprenavir gavage and aerosol and darunavir aerosol prevent pepsin-mediated laryngeal damage in vivo. Representative specimens at 400×. Solvent control group laryngeal epithelium was characterized by a single layer of respiratory epithelium with no reactive changes. In mice treated with pepsin-pH7, the laryngeal epithelium exhibited reactive epithelial changes and apoptotic debris. Fosamprenavir gavage and aerosol protected against pepsin-mediated laryngeal damage as indicated by normal histology in mice receiving fosamprenavir gavage or aerosol with saline (solvent), or fosamprenavir gavage or aerosol with pepsin-pH7. Darunavir gavage elicited mild reactivity (rare intraepithelial lymphocytes) in the saline treatment group; the darunavir gavage group with pepsin-pH7 appeared similar. Darunavir aerosol provided mild protection against pepsin-mediated damage. Epithelial injury was still present (mildly increased intraepithelial inflammatory cells and reactive epithelial cells), however no apoptosis was observed. Scale bar=200 um.

FIG. 11 is a certificate of analysis for 1 kg.

FIG. 12 is a certificate of analysis for 250 g.

### DETAILED DESCRIPTION

In the present application, the inventors disclose a novel means to treat reflux conditions, including GERD, airway reflux such as laryngopharyngeal reflux (LPR). The deleterious changes in the laryngopharynx observed in LPR develop following direct contact of the mucosa with refluxed gastric contents, which consist of acid as well as pepsin, bile, and pancreatic enzymes.

The present application provides oral alginate formulations that provide sustained release in a subject, reducing one or more symptom of reflux conditions. This new approach would be amenable to local treatment of readily accessible airways affected by LPR allowing lower dosing, the advantage of which is self-evident in that targeted delivery would simultaneously increase efficacy and limit systemic side effects.

Herein, therapeutic compounds were screened for pepsin binding and inhibition. Specific HIV protease inhibitors that inhibited pepsin were administered orally and by inhalation in an LPR mouse model to assess their potential for the treatment of LPR. Oral alginate formulations that provide extended release were developed that provides the most effective delivery.

Local esophageal treatment of GERD has been an area of interest for several decades, however drug delivery to the esophagus is challenging due to the exceedingly short esophageal transit time of orally administered drugs (<16s even when supine). Thickening agents and mucoadhesive substances that prolong esophageal retention and contact time of liquid suspensions have been investigated to overcome this limitation with efforts to date predominantly focused on substances that coat the esophagus and provide topical protection against refluxed acid, termed “esophageal bandages” by Potts et al. This research led to numerous patents for mucoadhesive carbonate formulations (reviewed by Batchelor 2005). Fosamprenavir retained in the esophagus would inactivate extracellular, mucosal-bound pepsin deposited during reflux; its absorption by the mucosa, facilitated by prolonged contact time would also inactivate endocytosed, intracellular pepsin. The desired formulation would permit esophageal absorption of a fraction of total dosage without dramatically impeding systemic delivery via the intestine.

Viscous and mucoadhesive formulations for esophageal retention have been investigated for local delivery of drugs for diagnosis of Barrett's esophagus, treatment of esophageal cancer and candidiasis and treatment of GERD and related pain and inflammation. Among mucoadhesive excipients, alginate has emerged as an optimal additive for prolonged esophageal residence time of liquid and solid pharmaceutical formulations.

Alginate is an extensively used bioadhesive polymers for drug delivery as it is non-toxic, biocompatible, non-immunogenic, biodegradable, mucoadhesive, readily available and cost-effective. The U.S. FDA recognizes alginate as “Generally Referred As Safe” (GRAS), or safe for alimentary use by qualified experts, listed in the Code of Federal Regulations Title 21 parts 182 and 184. Its pH-dependent gelation and chemical versatility, amenable to modifications to tailor its properties, has made alginate one of the most extensively explored mucoadhesive biomaterials, finding application in various modified drug delivery systems such as hydrogels, microparticles, nanoparticles and adhesive tablets and films for buccal drug delivery.

Our research demonstrated the assessment of the in vitro esophageal retention characteristics of several widely utilized pharmaceutical coating materials for solid pharmaceutical formulations. We found sodium alginate (1.5% w/w; medium viscosity grade from Mactocystis pyrifera) provided superior esophageal adhesion which was capable of ‘self-repairing’ (re-adhering at the next contact point if dislodged). Batchelor et al. demonstrated that alginate (at 2% w/v in 1 ml dose, i.e. 0.02 mg) adheres to the esophageal mucosa in vitro model for periods of up to 60 minutes. Using excised porcine esophagus with saliva wash simulated by constant wash of human saliva at 1 ml/min, we found that lower molecular weight alginates (<75 kDa) bearing lower viscosity (<0.02 Pa s), demonstrated significantly lower esophageal retention than others tested, while alginates with high viscosity (>2.93 Pa s) demonstrated greater esophageal retention at 3 min but similar retention as mid-range viscosity alginates at 15 or 30 min. G/M ratio of alginates had no effect on esophageal adhesion. An alginate of mid-range molecular weight (240 kDa), viscosity (0.51 Pa s) and G/M ratio (44/56) exhibited 21.9±9.5% retention at 30 min when provided at 0.02 mg in 1 ml dose (in water).

Sodium alginate of medium viscosity provided at the MDE of 24.5 mg per 10 ml dose b.i.d. in our formulation would therefore be predicted to confer 20% retention of bound drug (and retention of ˜5 mg alginate) in the esophagus for at least 30 minutes.

### Therapeutic Benefit for LPR

Higher doses of alginate in alginate-antacid medications (1000 mg per dose) have a long history of use as a monotherapy for mild to moderate GERD and a complimentary therapy for breakthrough symptoms of those taking PPIs. The therapeutic benefit of alginate is primarily attributed to its raft-forming activity. The alginate raft floats over stomach contents thereby displacing the postprandial acid pocket near the gastroesophageal junction, and effectively reducing acidic reflux events. Secondary mechanisms of anti-reflux activity are thought to be conferred by its mucoadhesion, which prevents diffusion of pepsin and acid such to underlying esophageal mucosa and concomitant epithelial barrier dysfunction, and enzymatic inhibition of pepsin. Notably, Chater et al. found that mid-range viscosity alginate (LF120) at just 0.68 mg/ml inhibited pepsin by 28.46±10.68% (at 1.36 mg/ml inhibition was 44.73±10.98). While this suggests that excipient alginate at the MDE (2.45 mg/ml b.i.d.) could have anti-peptic activity, the therapeutic value of alginate at such low concentrations is unlikely given numerous clinical trials and metanalysis reporting the therapeutic benefit of anti-reflux medications containing 1000 mg sodium alginate per 10 ml dose (Gaviscon Advance, Reckitt Benckiser, Slough, UK), relative to viscosity-matched placebo or products intended primarily as antacids which provided only transient symptom relief regardless of presence of alginate at excipient concentration (Gaviscon tablet or liquid antacid, GlaxoSmithKline Consumer Healthcare, Pennsylvania). The lack of therapeutic benefit of the latter is thought to be due to their inability to form coherent rafts.

Alginate-based anti-reflux medications have recently demonstrated therapeutic efficacy for throat symptoms of LPR. As for esophageal symptoms, therapeutic activity is attributed to raft-formation in the stomach, particularly given improbable contact between orally administered alginate and the larynx. McGlashan et al.²⁷ examined the efficacy of alginates for LPR symptoms and endoscopic findings in 49 patients with RSI and RFS-confirmed LPR randomized to receive liquid alginate suspension (n=24; 10 ml, four times daily post meal and at bedtime; Gaviscon Advance) or no treatment (n=25; control): pre-treatment mean (SD) RSI and RFS scores were similar for the treatment group (23.9 (7.0) and 10.4 (3.6)) and controls (24.6 (7.4) and 10.3 (3.3)), however alginate treatment improved LPR symptoms and findings as indicated by significant differences between treatment and control group RSI at 2-months (11.2 (7.0) vs. 16.8 (6.4), P=0.005) and 6-months (11.2 (8.1) vs. 18.3 (9.4), P=0.008) and RFS at 6-months (7.1 (2.8) vs. 9.5 (3.4), P=0.005). Similarly, in a study of personalized treatments for LPR subtypes (acid to alkaline), Lechien et al.²⁶ found that an anti-reflux diet and thrice daily post meal alginate (Gaviscon Advance) or magaldrate (Riopan, Takeda, Zaventem, Belgium) improved voice quality of patients with HEMII-pH testing-confirmed alkaline LPR (n=48) as indicated by reduced dysphonia and roughness scores (GRBAS scale) and improved jitter, shimmer and noise to harmonic ratio (Lechien et al. 2021).

The present invention provides a sustained-release formulation of oral fosamprenavir, using sodium alginate toincrease muco-adhesion and prolong drug delivery in the esophagus, that will improve esophageal symptoms in the 25-50% LPR patients that also have GERD, and thus have superior efficacy over oral fosamprenavir/Lexiva.

In one embodiment, the formulation contains a low excipient level dose of sodium alginate to prolong drug delivery to the esophagus by increasing muco-adhesion. This is expected to benefit pepsin-mediated esophageal inflammation, mucosal damage and associated symptoms. While high doses of alginate are expected to have therapeutic benefit due to raft formation, the present lower dose formulations are expected to increase muco-adhesion to prolong esophageal retention.

In-vitro tests for esophageal retention will include a texture analyzer for muco-adhesion.

Studies using combined multichannel intraluminal impedance with pH (MII-pH) monitoring have shown that many episodes of LPR are nonacidic, and that weakly and nonacidic reflux is associated with persistent symptoms in acid-suppressed patients (39-42). Pepsin, the chief digestive enzyme in the stomach, has been increasingly implicated as contributing to the damage and inflammation associated with LPR (17-23). Importantly, while the stomach and esophagus have internal defense mechanisms against pepsin, such as mucus, peristalsis, and bicarbonate secretion, laryngeal tissues do not (26). In the airways, which have a neutral pH (below 8), pepsin is enzymatically inactive but stable. However, when pepsin is taken up by laryngeal and hypopharyngeal cells via receptor-mediated endocytosis, it is retained in intracellular vesicles of low pH where it is presumed to be reactivated and cause damage (20, 32, 33, 49, 52). While many episodes of LPR are weakly acidic or nonacidic, pepsin is present in all refluxate (24), and is frequently detected in airway tissue and secretions from patients with LPR. For example, the inventors have demonstrated that endocytosed nonacidic pepsin induces expression of proinflammatory cytokine genes in hypopharyngeal cells. This response is similar to the response that occurs in reflux esophagitis, which contributes to disease severity in GERD patients (21, 31). Importantly, inhibition of pepsin's proteolytic activity (i.e., using pepstatin, curcumin, ecabet sodium, anthocyanin, or pre-incubation at pH 8.0 before decreasing the pH to 7.0) has been shown to abrogate this damage and inflammation (5, 7, 22, 33, 52, 54-56), making pepsin a promising therapeutic target for the treatment of airway reflux.

The present inventors believe that LPR is more dependent on pepsin-mediated damage than on acid-mediated damage, and that drugs that specifically target pepsin should be effective for patients with nonacid reflux. These drugs could finally provide a treatment option for patients who are refractory to proton pump inhibitors (PPI). Pepsin can be inhibited by two mechanisms: (1) via irreversible inactivation, which prevents it from becoming reactivated inside intracellular compartments of lower pH, and (2) via a receptor antagonist, which prevents pepsin uptake by receptor-mediated endocytosis. While the pepsin inhibitor pepstatin is already commercially available, it has poor water-soluble characteristics and pharmacokinetic properties. Thus, new pepsin inhibitor compounds with greater bioavailability are needed.

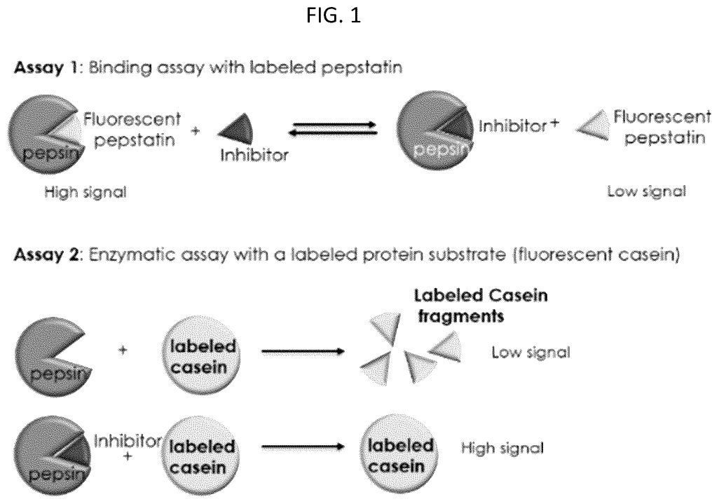

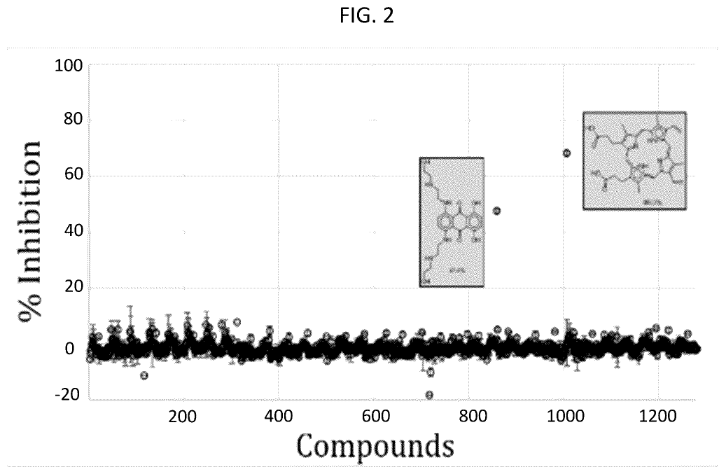

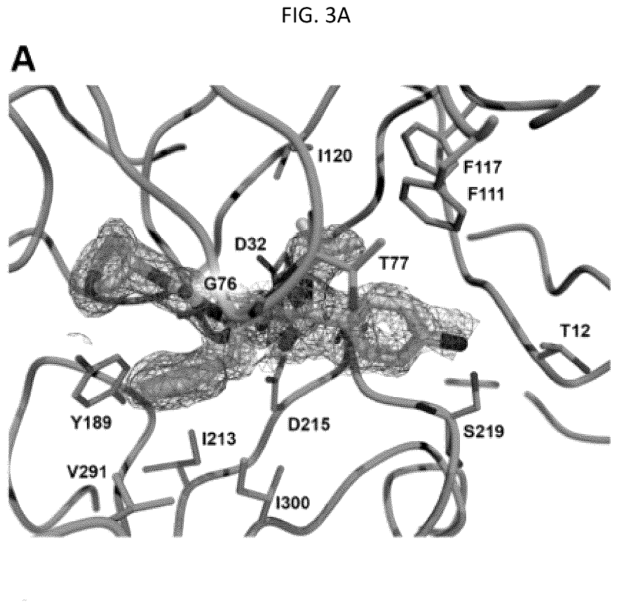

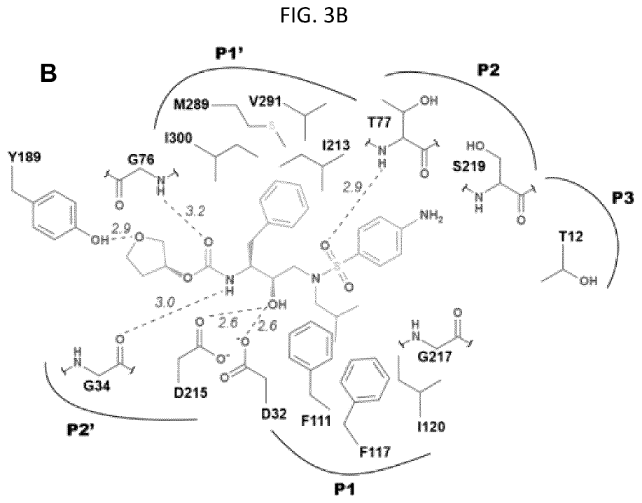

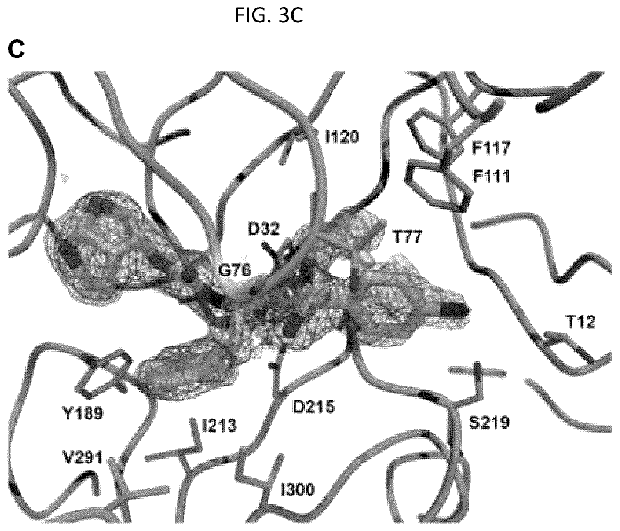

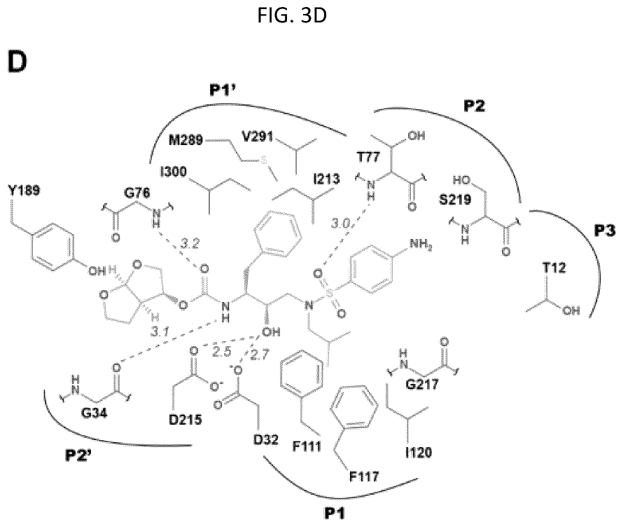

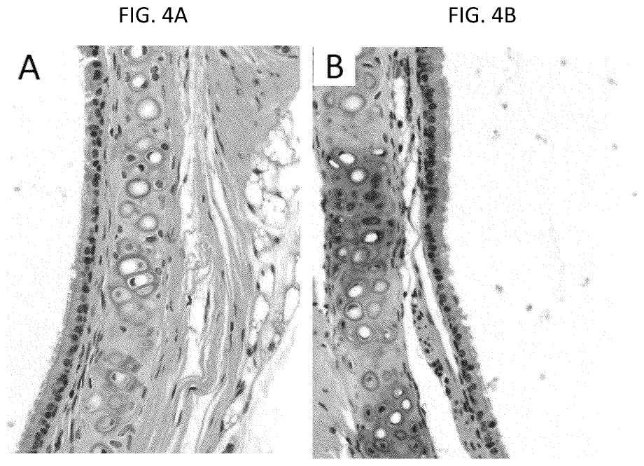

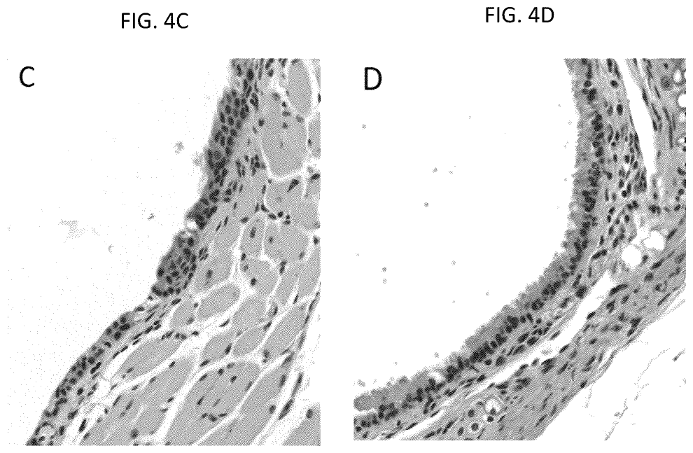

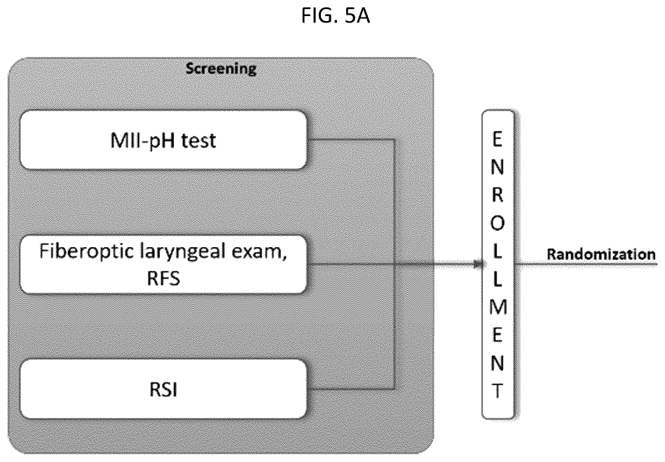

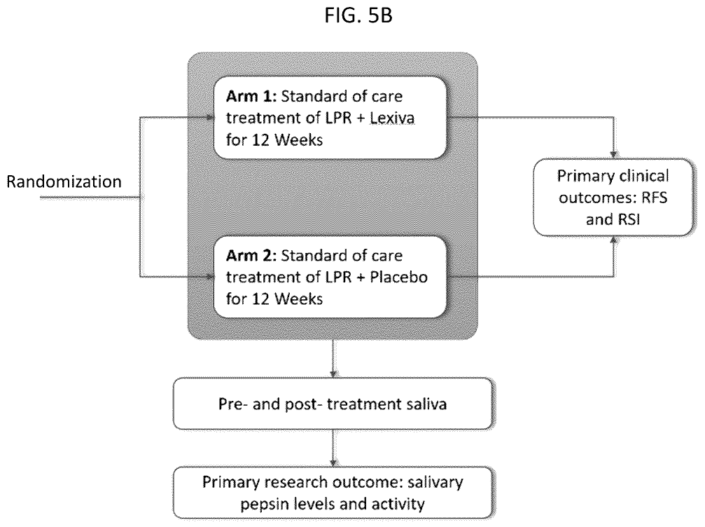

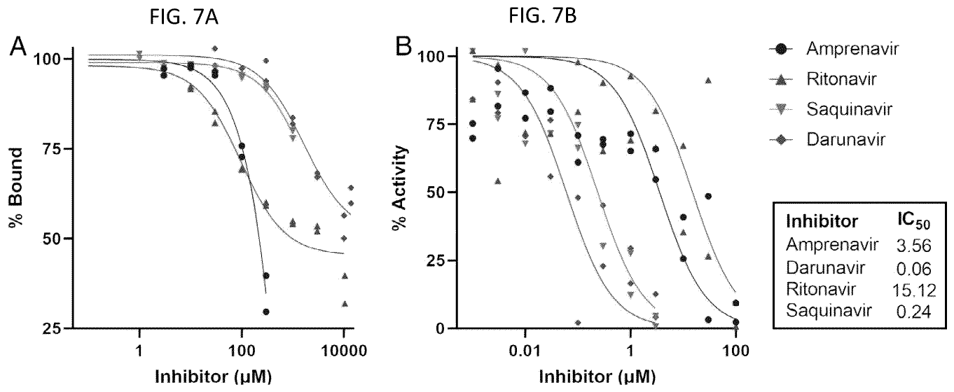

In the present application, the inventors screened therapeutic compounds for their ability to bind to pepsin and inhibit its enzymatic activity and identified specific HIV protease inhibitors with these abilities (see Example 1). Several HIV protease inhibitors have already been approved by the U.S. Food and Drug Administration (FDA) for the treatment of HIV, making these drugs ideal candidates to test the efficacy of pepsin inhibition for the treatment of LPR. Using epidemiological data, the inventors demonstrated that patients taking HIV protease inhibitors have a significantly lower incidence of airway reflux (0.2%) compared to the general population (10-34.4%), supporting the idea that these HIV drugs might be repurposed to treat LPR. Of the ten commercially available HIV protease inhibitors, the inventors determined that four (i.e., amprenavir, darunavir, ritonavir, and saquinavir) have the ability to bind to and inhibit pepsin activity in vitro (FIGS. 7A and 7B). To test these drug candidates in vivo, the inventors established a novel mouse model of LPR (FIG. 1). These mice will be used to test the ability of HIV protease inhibitors to ameliorate pepsin-mediated laryngeal mucosal damage and inflammation. The mice are given HIV protease inhibitors both by oral gavage and by aerosolized delivery to compare the results of systemic and local delivery, respectively. Building on the results of these animal studies, the inventors will test the efficacy of promising HIV protease inhibitors in a 12-week randomized, double blind, placebo-controlled clinical trial (FIGS. 5A and 5B).

### Methods:

The present invention provides methods of treating reflux in a subject in need thereof, preferably airway reflux. The methods involve administering a therapeutically effective amount of an formulation comprising an HIV protease inhibitor and alginate to a subject to treat the reflux. As used herein, the term “airway reflux” refers to inflammation of the upper and lower airways caused by reflux of gastric contents. The term airway reflux is used interchangeably with the alternative terms “supraoesophageal reflux” and “extraoesophageal reflux.” These broad terms encompass several related reflux conditions, which include gastropharyngeal reflux (GPR; the backflow of gastric contents up to the esophagus), laryngopharyngeal reflux (LPR; the backflow of gastric contents beyond the esophagus into the laryngopharynx), and esophagopharyngeal reflux (EPR; a similar condition to LPR that is characterized by esophageal abnormalities). Reflux also includes gastroesophageal reflux disease (GERD) which refers to irritation of the esophagus caused by reflux of stomach's contents back up into the esophagus. The reflux treated herein is preferably GERD patients that are refractory to protein pump inhibitor (PPI) therapy.

As used herein, the term “HIV protease inhibitor” refers to any antiviral drug that inhibits one or more HIV proteases. HIV protease inhibitors prevent viral replication by selectively binding to HIV proteases and blocking proteolytic cleavage of protein precursors that are necessary for the production of infectious viral particles. Suitable HIV protease inhibitors include those that have been approved by the Food and Drug Administration (FDA) for the treatment of HIV, including amprenavir (IUPAC: [(3S)-oxolan-3-yl] N-[(2S,3R)-4-[(4-aminophenyl)sulfonyl-(2-methylpropyl)amino]-3-hydroxy-1-phenylbutan-2-yl]carbamate), ritonavir (IUPAC: 1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl]carbamoyl]amino]butanoyl]amino]-1,6-diphenylhexan-2-yl]carbamate), lopinavir (IUPAC: (2S)-N-[(2S,4S,5S)-5-[[2-(2,6-dimethylphenoxy)acetyl]amino]-4-hydroxy-1,6-diphenylhexan-2-yl]-3-methyl-2-(2-oxo-1,3-diazinan-1-yl)butanamide), saquinavir (IUPAC: (2S)-N-[(2S,3R)-4-[(3S,4aS,8aS)-3-(tert-butylcarbamoyl)-3,4,4a,5,6,7,8,8a-octahydro-1H-isoquinolin-2-yl]-3-hydroxy-1-phenylbutan-2-yl]-2-(quinoline-2-carbonylamino)butanediamide), nelfinavir (IUPAC: (3S,4aS,8aS)-N-tert-butyl-2-[(2R,3R)-2-hydroxy-3-[(3-hydroxy-2-methylbenzoyl)amino]-4-phenylsulfanylbutyl]-3,4,4a,5,6,7,8,8a-octahydro-1H-isoquinoline-3-carboxamide), darunavir (IUPAC: [(3aS,4R,6aR)-2,3,3a,4,5,6a-hexahydrofuro[2,3-b]furan-4-yl]N-[(2S,3R)-4-[(4-aminophenyl)sulfonyl-(2-methylpropyl)amino]-3-hydroxy-1-phenylbutan-2-yl]carbamate), indinavir ((2S)-1-[(2S,4R)-4-benzyl-2-hydroxy-5-[[(1S,2R)-2-hydroxy-2,3-dihydro-1H-inden-1-yl]amino]-5-oxopentyl]-N-tert-butyl-4-(pyridin-3-ylmethyl)piperazine-2-carboxamide), atazanavir (IUPAC: methyl N-[(2S)-1-[2-[(2S,3S)-2-hydroxy-3-[[(2S)-2-(methoxycarbonylamino)-3,3-dimethylbutanoyl]amino]-4-phenylbutyl]-2-[(4-pyridin-2-ylphenyl)methyl]hydrazinyl]-3,3-dimethyl-1-oxobutan-2-yl]carbamate), tipranavir (IUPAC: N-[3-[(1R)-1-[(2R)-4-hydroxy-6-oxo-2-(2-phenylethyl)-2-propyl-3H-pyran-5-yl]propyl]phenyl]-5-(trifluoromethyl)pyridine-2-sulfonamide), and cobicistat (IUPAC: 1,3-thiazol-5-ylmethyl N-[(2R,5R)-5-[[(2S)-2-[[methyl-[(2-propan-2-yl-1,3-thiazol-4-yl)methyl]carbamoyl]amino]-4-morpholin-4-ylbutanoyl]amino]-1,6-diphenylhexan-2-yl]carbamate). The HIV protease inhibitor used with the present invention should be capable of binding to and inhibiting the enzymatic activity of pepsin. Thus, in some embodiments, the HIV protease inhibitor is amprenavir, darunavir, ritonavir, or saquinavir, which were shown to bind to and inhibit pepsin in Example 1. In some embodiments, the HIV protease inhibitor is amprenavir (IUPAC: [(3S)-oxolan-3-yl] N-[(2S,3R)-4-[(4-aminophenyl)sulfonyl-(2-methylpropyl)amino]-3-hydroxy-1-phenylbutan-2-yl]carbamate) or its prodrug fosamprenavir (IUPAC: [(3S)-oxolan-3-yl] N-[(2S,3R)-4-[(4-aminophenyl)sulfonyl-(2-methylpropyl)amino]-1-phenyl-3-phosphonooxybutan-2-yl]carbamate). HIV protease inhibitors are known in the art and commercially available.

Fosamprenavir is a prodrug of amprenavir that is marketed by ViiV Healthcare as a calcium salt under the trade names Lexiva (U.S.) and Telzir (Europe). The body must metabolize fosamprenavir to form amprenavir, which is the active form of the drug. Thus, administering amprenavir as a prodrug prolongs the duration of time that it is available in the body, acting like a slow release formulation. Further, fosamprenavir has shown excellent pharmacokinetics in mice and because it is already FDA approved, fosamprenavir could be fast-tracked into a pilot clinical trial. In some embodiments, the HIV protease inhibitors for use in the compositions and methods described herein have an IC₅₀ in the micromolar range (μm). In some preferred embodiments, the HIV protease inhibitors for use in the compositions and methods described herein have an IC₅₀ in the nanomolar (nm) range.

In the present methods, the HIV protease inhibitor may be administered using any route that is effective for the treatment of reflux, preferably airway reflux, and preferably provides a formulation for oral administration. As used herein, the terms “administering” and “administration” refer to any method of providing a pharmaceutical preparation to a subject. Such methods are well known to those skilled in the art and include, but are not limited to, oral administration. Administration can be continuous or intermittent.

In some embodiments, the HIV protease inhibitor is administered orally for the treatment of the reflux. For example, in some embodiments, the HIV protease inhibitor is administered twice daily at about 0.7-1.4 g (i.e., a dosage that is FDA-approved for the treatment of HIV and thus safe).

The methods of the present invention are used to treat to reflux in a subject in need thereof. In some embodiments, the reflux may be airway reflux. In other embodiments, the reflux may be GERD, preferably GERD in a subject that is refractory to proton pump inhibition. As used herein, the term “subject in need thereof” or “patient” refers to any human or animal suffering from reflux. In some embodiments, the subject has an airway reflux. In some embodiments, the airway reflux condition selected from laryngopharyngeal reflux (LPR), gastropharyngeal reflux (GPR), and esophagopharyngeal reflux (EPR). In some embodiments, the subject is a subject with reflux episodes caused by weakly acidic or nonacidic reflux. In another embodiment, the subject is a subject refractory to proton pump inhibitor (PPI) therapy.

As used herein, the terms “treat”, “treating” or “treatment” describes the management and care of a subject for the purpose of combating a disease, condition, or disorder. Treating includes the administration of protease inhibitor or composition of present invention to prevent the onset of the symptoms or complications, to alleviate the symptoms or complications, or to eliminate the disease, condition, or disorder. In preferred embodiments, the methods and compositions of the present reduce mucosal damage and inflammation in the airway of the subject. Treatment also includes reducing one or more symptoms of airway reflux, suitably LPR, GPR or ERP, for example, reduction of chronic cough, throat clearing, postnasal drip, hoarseness or dysphonia, globus sensation, dysphagia, dyspnea, or combinations thereof. Treatment also includes reducing chronic laryngeal irritation and inflammation. Treatment in one embodiment also includes reducing one or more symptom of GERD that is refractory to PPI, for example, reducing one or more of the following symptoms: a burning sensation in your chest (heartburn), usually after eating, which might be worse at night, chest pain, difficulty swallowing, regurgitation of food or sour liquid, sensation of a lump in your throat, among others.

The terms “effective amount” or “therapeutically effective amount” refer to an amount sufficient to effect beneficial or desirable biological or clinical results. That result can be reducing, alleviating, inhibiting or preventing one or more symptoms of a disease or condition, reducing, inhibiting or preventing laryngeal irritation, reducing or inhibiting laryngeal irritation or mucosal damage, or reducing, alleviating, inhibiting or preventing one or more symptoms of airway reflux, or any other desired alteration of a biological system. In some embodiments, the effective amount is an amount suitable to provide the desired effect, e.g., reduce mucosal damage and inflammation in the airway. The response to a treatment of airway reflux may be assessed using any standard clinical method including, without limitation, visual inspection of the larynx (e.g., fiberoptic laryngeal exam), a reflux symptom index (RSI), reflux finding score (RFS) (e.g., physician reported score based on visual inspection of the larynx), combined esophageal multichannel intraluminal impedance and pH monitoring (MII-pH), reflux symptom score (RSS), reflux sign assessment (RSA), or pepsin activity within the saliva. Alternatively, the response to a treatment of airway reflux may be assessed using by evaluating the inflammation in a tissue sample taken from the airway of the subject, for example, by hematoxylin and eosin (H&E) staining or by detection of the presence of neutrophil infiltrate, keratinization, and necrosis. Another suitable method is measure pepsin activity pre and post 12-week treatment. While it is not expected that the HIV inhibitor will prevent reflux or affect pepsin protein levels, it will inactivate the pepsin enzyme, therefore measuring pepsin activity in saliva post-treatment would confirm that the treatment is inactivating pepsin in the airway. This is currently a research tool to assess efficacy in vivo.

Patients with reflux episodes caused by weakly acidic or nonacidic reflux are largely refractory to proton pump inhibitor (PPI) therapy, which suppresses acid production but does not affect pepsin activity. The methods of the present invention will be of particular benefit to this group of refractory patients, who are in desperate need of an alternative to PPIs. As used herein, the phrase “refectory to treatment” refers to a condition that does not respond to treatment. For example, a patient's reflux may be deemed refractory to PPI therapy if a three-month long, twice-daily treatment with a PPI fails to improve the condition substantially. The response to a treatment of reflux may be assessed using any standard means known in the art including, without limitation, a reflux symptom index (RSI), reflux finding score (RFS), combined esophageal multichannel intraluminal impedance and pH monitoring (MII-pH), reflux symptom score (RSS), or reflux sign assessment (RSA). See the Examples section for a more detailed description of these measures. For example, an effective treatment would decrease the RSI and/or the RFS to normative values, e.g., RSI≤13, RFS≤7 or a combination thereof.

### Compositions:

The present invention also provides compositions comprising an oral formulation of an HIV protease inhibitor and an alginate and a pharmaceutically acceptable carrier. Commercially available HIV protease inhibitors are commonly formulated as tablets or oral suspensions for systemic drug delivery. For example, in some embodiments, the composition is formulated for oral administration.

The compositions of the present invention may include any pharmaceutically acceptable carrier that allows for oral delivery. “Pharmaceutically acceptable carriers” are known in the art and include, but are not limited to, for example, suitable diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

The compositions of the present invention may further include additional components to influence the physical state, solubility, stability, rate of in vivo release, and rate of in vivo clearance of the HIV protease inhibitor. Suitable components include, without limitation, buffers (e.g., Tris-HCl, acetate, phosphate), additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances, and tonicity modifiers (e.g., lactose, mannitol). Additionally, the compositions may be formulated for controlled or sustained release of the HIV protease inhibitor, for example, via formulation in lipophilic depots (e.g., fatty acids, waxes, oils).

The composition of the present invention may further include a suspending agent, a preservative, a sweetener, a flavoring, water, and a combination thereof. Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate. Preparations for oral administration may also be suitably formulated to give controlled release of the compound, as is well known.

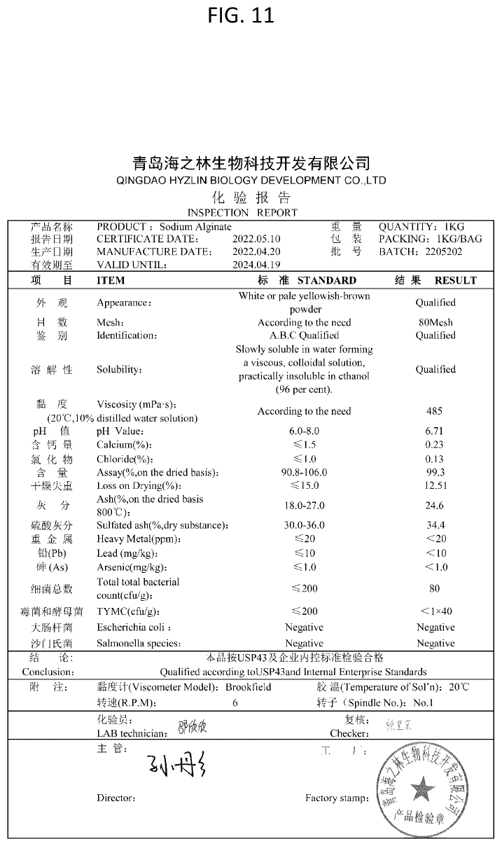

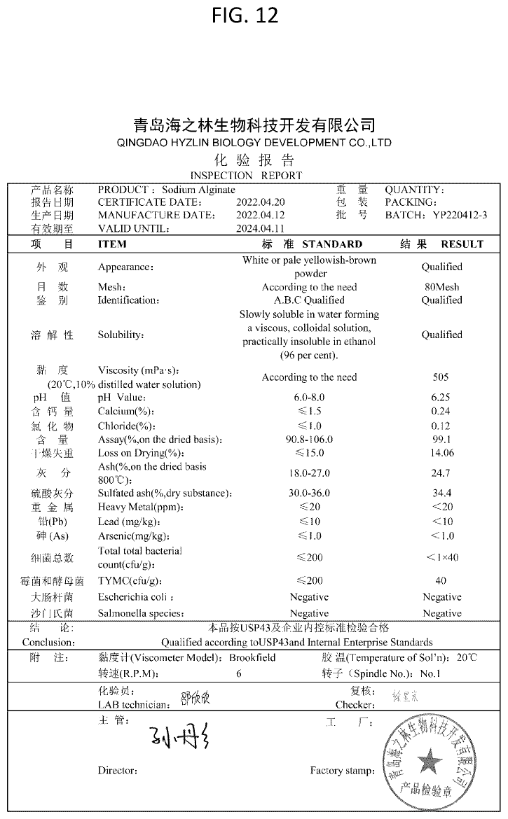

Table 1 provides a proposed formula. It is expected that the API amount will be about 20-30% of the total weight of the dry product formulation. FIGS. 11 and 12 are certificates of analysis for the composition described herein.

TABLE 1
Proposed Formulation

| | Mg/10 mL (after |
|---|---|
| Ingredient | reconstitution) |
| Fosamprenavir | 1200-1500 (e.g., 1400) |
| Sodium Alginate | 20-30 (e.g. 24.5) |
| Optional additional ingredients | |
| Suspending Agent (e.g., Hypromellose, Xantham | |
| Gum) | |
| Preservative(s) (e.g., Methylparaben, | |
| Propylparaben, Sodium Benzoate) | |
| Sweetener (e.g., Sucralose, acesulfame) | |
| Flavor (e.g, cherry) | |
| Purified Water | |

The compositions may be prepared in unit dosage forms for administration to a subject. The amount and timing of administration are at the discretion of the treating clinician to achieve the desired outcome.

The HIV protease inhibitor included in the compositions of the present invention may be any HIV protease inhibitor that is suitable for the treatment of airway reflux, as discussed above. In some embodiments, the HIV protease inhibitor included in the composition is amprenavir, darunavir, ritonavir, saquinavir, or a derivative thereof. In preferred embodiments, the HIV protease inhibitor is amprenavir or its prodrug fosamprenavir. In another embodiment, the HIV protease inhibitor is darunavir.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The references cited herein are explicitly incorporated by reference in their entireties.

The invention will be more fully understood upon consideration of the following non-limiting examples.

### EXAMPLES

### Example 1: Oral and Inhaled Fosamprenavir Reverses Pepsin-Induced Damaged in a Laryngopharyngeal Reflux Mouse Model

Reference is made to Johnston, N., Samuels, T. L., Goetz, C. J, Arnold, L. A., Smith, B. C., Seabloom, D., Wuertz, B., Ondrey, F., Wiedmann, T. S., Vuksanovic, N., Silvaggi, N. R., MacKinnon, A. C., Miller, J., Bock, J. and Blumin, J H. (2022), Oral and Inhaled Fosamprenavir Reverses Pepsin-Induced Damage in a Laryngopharyngeal Reflux Mouse Model. The Laryngoscope. and is incorporated by reference.

### Methods

### Binding and Activity Assays

To examine whether HIV protease inhibitors bound and inhibited pepsin, we developed assays based on fluorescence polarization which measures size-dependent molecular rotation thereby permitting detection of degradation, association and dissociation events⁸⁰. A competitive binding assay was designed employing pepstatin, an inhibitor of sub-nanomolar affinity⁸¹. Pepstatin-Alexa647 was synthesized by dissolving 1 mg pepstatin A (Sigma-Aldrich) in a 50:50 mixture of dimethylformamide (DMF) and dimethylsulfoxide (DMS) followed by the addition of N,N,N′,N′-Tetramethyl-O—(N-succinimidyl)uronium tetrafluoroborate (0.6 mg) and trimethylamine (10 μL) DMF. The mixture was stirred for 1 hour, after which 1 mg Alexa Fluor 647 Cadaverine, Disodium Salt (ThermoFisher Scientific) was added. After 2 hours, the solvents were evaporated under high vacuum (35° C.) and residue partially dissolved in 10% methanol and transferred onto a C18 cartridge (Waters Corporation, Milford, MA). Increasing percentages of methanol were used for the elution. Pepstatin-Alexa647 eluted at 45% methanol. An enzymatic inhibition assay was designed using casein substrate.⁸² Bovine alpha casein (Sigma-Aldrich, St. Louis, MO) was labeled with Alexa Fluor 647 Carboxylic Acid, Succinimidyl Ester (ThermoFisher Scientific, Waltham, MA) as described.⁸² Briefly, the two were combined at 2.5 ug/mg label to protein ratio in 0.1M sodium bicarbonate for 15 minutes and labeled casein was separated from unbound label in a Sephadex G-25 (Sigma-Aldrich) column comprised of 90×5 mm packed beads in a glass Pasteur pipette, eluted with dPBS pH7.4 (ThermoFisher Scientific). The fast-moving band (casein-bound fluorophore) was collected in ˜0.4 ml volume. Concentration of resultant probe (casein-Alexa647 in PBS-azide) was estimated via spectrophotometry using Beer's law (Implen Nanophotometer, Implen, Inc. Westlake Village, CA).

Assays were optimized using ranges of 0.3-1000 μM unlabeled pepstatin, 100-500 nM pepstatin-Alexa647 or casein-Alexa647 probe, 0.003-3 U/μl porcine pepsin (Worthington Biochemical Corporation, Lakewood, NJ), and 5-37.5% DMSO (HIV protease inhibitor diluent) in 0.1 M HCl, pH 1 with 0.01% v/v Tween-20 in 20 μl volumes in 384-well black optical plates (Nunc, Roskilde, DK) and read on a BioTek Cytation 5 (BioTek Instruments, Winooski, VT) with far red FP filter cube (excitation/emission 620/680 nm). Unlabeled pepstatin dose-response curves were used to ensure that the assays were responsive to pepsin inhibition. Conditions yielding maximal dynamic assay range were used to assess HIV protease inhibitors: 100 nM probe, 0.03 U/ul porcine pepsin A, 37.5% DMSO for competitive binding assay, and 200 nM probe, 0.01 U/μL pepsin, 5% DMSO for peptic activity assay. The HIV protease inhibitors (amprenavir, ritonavir, lopinavir, saquinavir mesylate, nelfinavir mesylate hydrate, darunavir ethanolate, indinavir sulfate salt hydrate; all Sigma-Aldrich) were dissolved in DMSO and tested under optimized assay conditions over three logs concentration. Assays were performed twice with triplicate reactions read for five minutes and mean mP plotted against probe concentration (binding assay) or read at <2 minutes intervals over 30 minutes with mean mP of plotted over time (activity assay). Half maximal inhibitory concentration (IC₅₀) of inhibitors were calculated from kinetic traces analyzed using an online tool (https://icekat.herokuapp.com/icekat)⁸³. mP was normalized to blank (absent inhibitor) to derive percent bound or activity.

### Crystallization

Saturated solutions of HIV protease inhibitors (amprenavir, ritonavir and darunavir ethanolate) were prepared in DMSO and centrifuged for 10 minutes at 31,000 rcf Supernatants were added to pepsin (200 mg/ml in water) at 1.6% (v/v) f.c. Due to poor solubility, a solvent for saquinavir mesylate was selected from the CryoSol screen (Molecular Dimensions, Holland, OH). CryoSol mixture SM2 (consisting of 37.5% v/v dioxane, 25% v/v DMSO, 12.5% v/v ethylene glycol, 12.5% v/v 1,2-propanediol, and 12.5% v/v glycerol) was selected as it provided both high solubility and protein compatible conditions for the co-crystallization mixture. Supernatant of saturated saquinivar solution in SM2 was combined with pepsin at 5% f.c. (v/v). Crystallization conditions were optimized by screening 200 mg/ml pepsin in the Salt RX screen (Hampton Research, Viejo, CA). Small bipyramid-shaped crystals formed in 3.5M ammonium chloride and 0.1 M sodium acetate trihydrate pH 4.6 after one week at room temperature served as microseed stock for co-crystallization with amprenavir, ritonavir and darunavir ethanolate per previously described methods⁸⁴. Diffraction quality crystals (triangular bi-pyramids, approximately 200×100×100 μm) formed after 2-7 days from hanging drops of 2 ul pepsin (180-210 mg/ml) and 1 ul microseed solution serially diluted 10-100× above 3-4M ammonium chloride and 0.1M sodium acetate trihydrate pH4.6. Crystals were cryoprotected by 30% glucose, 5M ammonium chloride and 0.1 M sodium acetate trihydrate pH4.6 and plunged in liquid nitrogen. Co-crystallization with saquinavir was performed in 0.1M acetic acid rather than sodium acetate trihydrate as this permitted large crystal formation without a microseed; crystals were cryoprotected by 30% w/v glucose, 5M ammonium chloride and 0.1 M sodium acetate trihydrate pH 4.6 and plunged in liquid nitrogen.

Diffraction datasets were collected at Life Sciences Collaborative Access Team (LS-CAT) beamlines at the Advanced Photon Source (APS), Argonne National Laboratory, equipped with MAR 300 CCD or Dectris Eiger 9M detectors and data were indexed, integrated and scaled using MOSFLM⁸⁵ or HKL2000⁸⁶.

Specifically, for pepsin:amprenavir, a 1.9 Å diffraction data set was collected at LS-CAT beamline 21-ID-F with a MAR 300 CCD detector using a 50×50 μm beam at a wavelength of 0.97872 Å. A total of 262 frames were collected from φ=0 to 130.5° with an oscillation range of 0.5° and detector distance of 250 mm. Exposure time was 0.5 seconds. Diffraction data were indexed, integrated and scaled using MOSFLM.

For pepsin:ritonavir, a 2.1 diffraction data set was collected at LS-CAT beamline 21-ID-D with Dectris Eiger 9M detector using a 50×50 μm beam at 1.12721 Å. 900 frames were collected from φ=0 to 180°, while oscillating at a rate of 1°/sec and slicing of 5 images/°. Crystal-to-detector distance was 160 mm. Diffraction data were indexed, integrated and scaled using MOSFLM.

For pepsin:darunavir, a 1.9 Å diffraction data set was collected at LS-CAT beamline 21-ID-G with MAR 300 CCD detector and 50×50 μm beam at 0.97856 Å. 900 frames were collected from φ=0 to 180° with an oscillation range of 0.2° and detector distance of 260 mm. Exposure time was 0.3 seconds. Diffraction data were indexed, integrated and scaled using HKL2000.

For pepsin:saquinavir, a 1.9 Å diffraction data set was collected at LS-CAT beamline 21-ID-F with MAR 300 CCD detector using a 50×50 μm beam at 0.97872 Å. 400 frames were collected from φ=20 to 100° with an oscillation range of 0.2° and detector distance of 200 mm. Exposure time was 0.5 seconds. Diffraction data were indexed, integrated and scaled using MOSFLM.

Initial phases were obtained by molecular replacement in PHASER⁸⁷. Unliganded porcine pepsin (PDB ID 4PEP) with B factors reset to 20.00 Å and solvent molecules removed was the search model. Model refinement was performed using phenix.refine (PHENIX⁸⁷⁻⁸⁹) and COOT^{90,91}. Geometric restraints for compounds were obtained from CCP4 monomer library⁹². Models were validated using MolProbity⁹³ as implemented in the PHENIX suite. Models of ritonavir and saquinavir were additionally optimized using PDB-REDO server ⁹⁴ prior to deposition. Electron density maps were generated via POVSCRIPT and POV-Ray and schematic representation by MarvinSketch,(http://www.ChemAxon.com) and Adobe Illustrator CC 2020.

### In Vivo Mouse Model

Experiments were approved by the University of Minnesota (UMN) Institutional Animal Care and Use Committee (1712-35415A) and performed at UMN. Three replicate animals per treatment condition were anticipated to suffice for verification of reproducibility in each experiment without excessive use of animal life. The three mice were randomly allocated to treatment groups. No data were excluded from analysis.

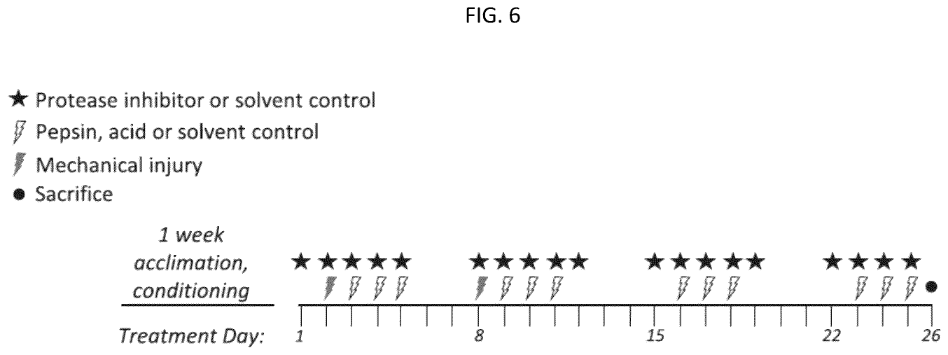

Six-week-old female Jackson A/J mice (Jackson Laboratory, Bar Harbor, ME) were fed D-62 powdered Wattenberg diet, 2 g/mouse/day⁹⁵ and allowed to acclimate for one week upon arrival prior to experiments. In accord with previously established methods for modeling aerodigestive tract damage attributed to GERD and LPR,^{1,95-99} mechanical injury applied during the first two weeks of a four-week treatment course was used to predispose the laryngeal mucosa to chemical injury by pepsin/acid applied throughout the four weeks. When performed in this manner, mechanical injury increases mucosal susceptibility to subsequent chemical injury while leaving little detectable injury at the conclusion of a four-week treatment course.⁹⁵ Mechanical injury was performed on all animals (including control) once weekly during the first two weeks of treatment as described (see experimental schema, FIG. 6)⁹⁵. Briefly, anesthetized mice were suspended by upper teeth on a slanted board under an operating microscope. Subglottis, glottis, and supraglottis were wounded under 6× magnification using a blunt, bent (135°) needle pulled distally to proximally making a mild abrasion.

In a preliminary experiment to validate the LPR mouse model (i.e. laryngeal damage by pepsin at neutral and acidic pH), 20 μl saline (solvent control) or 0.3 mg/ml pepsin at pH7.0 or 4.0 were provided to mice (n=3) by laryngeal instillation at 24, 48, and 72 hours after mechanical injury during weeks 1 and 2 (FIG. 6); laryngeal instillation without wounding (3 days/week) continued during weeks 3 and 4. Mice were anesthetized with 225-240 mg/kg intraperitoneal Avertin (2,2,2-Tribromoethanol) prior to each wounding and laryngeal instillation. Mice were sacrificed at conclusion of the fourth week.

To test the protective effect of HIV protease inhibitors on pepsin-mediated damage in vivo, inhibitors were delivered by aerosol or gavage concurrently with wounding (days 2, 8) and solvent/pepsin instillation (days 3-5, 9-11, 16-18 and 23-25). Aerosol or gavage was provided on days 1-5, 8-12, 15-19, and 22-25, and mice sacrificed day 26. Mice were anesthetized with isoflurane (3% in 2.5LPM, 3-5 minutes prior to procedures) as opposed to Avertin due to frequency. Lexiva and Prezista (hereafter referred to by generic: fosamprenavir and darunavir, respectively) were used for gavage, and respective pure drugs for aerosol (fosamprenavir from Anant Pharmaceuticals, Ambernath, Maharashtra India and darunavir from Ambeed, Arlington Heights, IL). Gavage dose was equivalent to that prescribed to HIV patients (20 mg/kg/day fosamprenavir; 8.6 mg/kg/day darunavir). Aerosol was generated as described¹⁰⁰. Briefly, a 10 ml suspension of drug in ethanol was placed in the baffle, such that the concentration would remain constant at the equilibrium solubility. Droplets of ethanol containing dissolved drug were generated by an ultrasonic atomizer (nominal frequency 1.7 MHz) and entrained by air at a flow rate of 0.5 LPM with a custom-built glass baffle (UMN Department of Chemistry Glass Shop). The aerosol cloud was then passed through a cylindrical drying column containing an annular ring of charcoal. The ethanol was removed and the emanating dry aerosol particles of pure drug were then directed into the exposure chamber. The mass deposited on filters was measured gravimetrically and total output rate (mg/min) was determined. The aerosol concentration (mass/volume of air) was calculated by dividing the total output rate by the air flow rate (0.5 LPM). The inhaled mass of drug (Mᵢₙₕ) for each mouse was defined as Mᵢₙₕ=[Aerosol]*RMV*t, where Aerosol is the aerosol concentration of drug, RMV is the respiratory minute volume of the mice (0.025 L/min), and t is the aerosol exposure time. Aerosol concentration was 0.09 mg/L fosamprenavir or 1.2 mg/L darunavir, therefore given the respiratory minute volume of mice (0.025 L/min), the inhaled mass was 0.93 mg/kg/day fosamprenavir or 12 mg/kg/day darunavir. Actual mass deposited was not determined but anticipated to be 10% of inhaled mass (the deposition fraction of 1 m aerosol particles in mice).

Tissues were collected, fixed in paraformaldehyde, embedded in paraffin and 4 um sections stained with hematoxylin and eosin (H&E) via automated stainer. Slides were reviewed by a board-certified pathologist (JM) blinded to treatment groups.

### Results

### Binding and Activity Assays

Four of the seven assayed HIV protease inhibitors bound and inhibited pepsin at low micromolar concentrations (FIGS. 7A and 7B): amprenavir, darunavir, ritonavir, and saquinavir. The in vitro activity of these four HIV protease inhibitors against pepsin provided the foundational support for further study.

### Structural Data

To aid interpretation of the in vitro binding and inhibition data, commercially available porcine pepsin (EC 3.4.23.1) was used for co-crystallization experiments to obtain structural data. Crystallization of human pepsin collected from volunteers failed presumably due to sample heterogeneity. Porcine pepsin shares 86% sequence identity with the human enzyme (PDB ID 1PSN)¹⁰¹ and its structure is nearly identical (root mean square deviation (RMSD) for all Cα atoms=0.50 Å). Minor differences in tertiary structure are localized to a loop of residues (277-282) which is not part of the binding cleft. Residues lining the active site cleft are highly conserved: of 17 making direct contact with inhibitors herein, just two differed (T12 and V291). Thus, porcine was deemed an acceptable substitute for human pepsin for assessing structural biology.

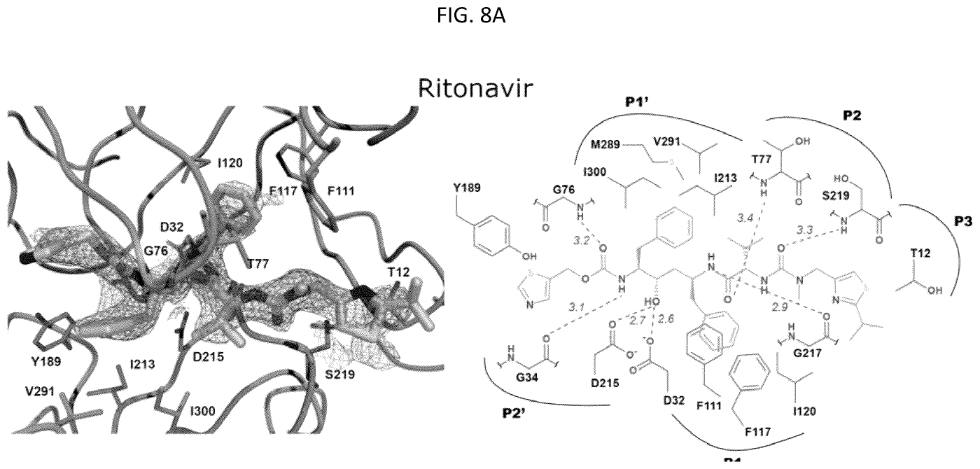

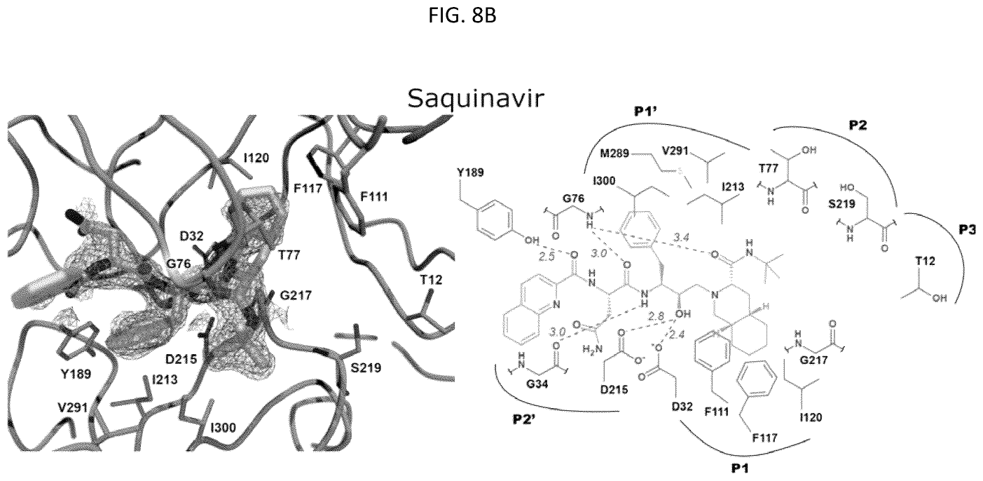

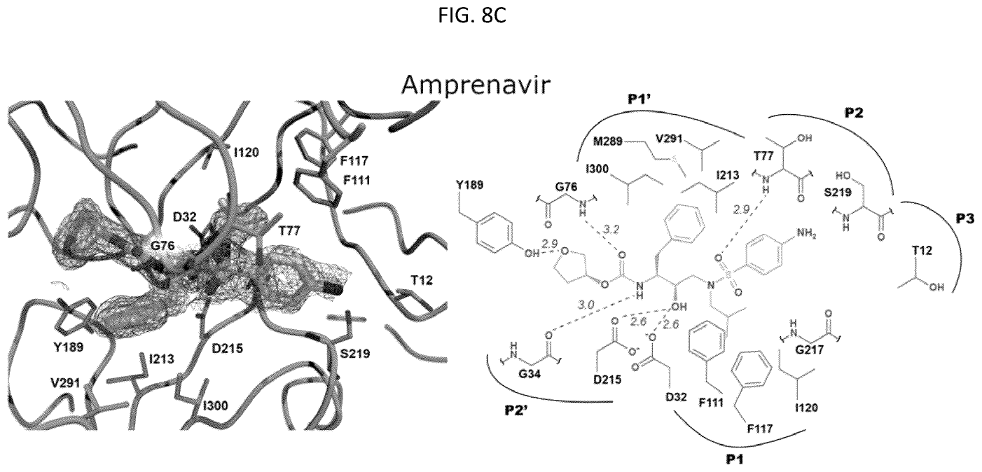

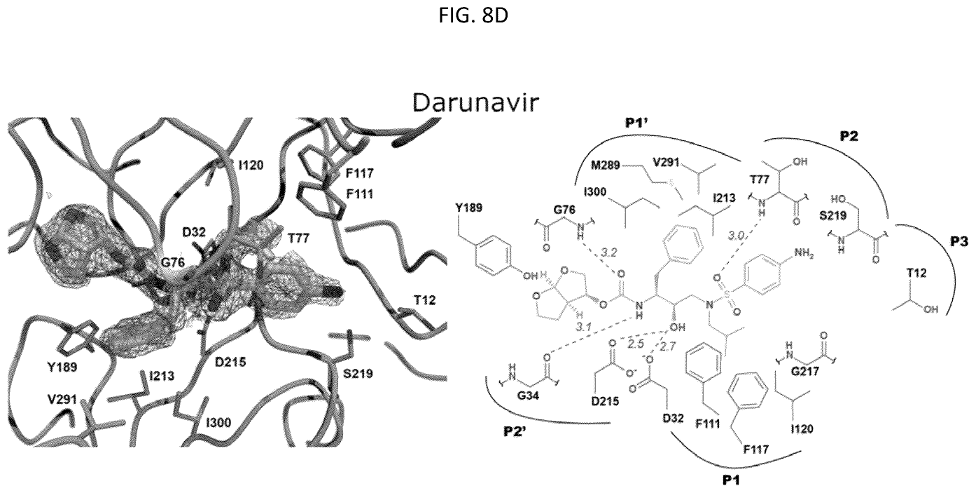

Porcine pepsin was co-crystalized amprenavir, darunavir, ritonavir, and saquinavir (Table 2 and FIGS. 8A-8D). All are peptidomimetics; the alcohol of the central phenylalaninol residue, which mimics the tetrahedral intermediate of peptide bond cleavage, is bound between catalytic aspartate residues, D32 and D215. Binding directionality of each (amino group of phenylalaninol on the prime side of the binding site) was the same as that for pepstatin¹⁰¹. Binding relied on van der Waals contacts between side chains of inhibitors and residues lining the binding site; few (5-6) hydrogen bonds were observed. For example, in the pepsin ritonavir complex (FIG. 8A), the β-homophenylalanine side chain is bound in the P1 subsite, making van der Waals contacts with F111, F117, and I120. The phenylalaninol side chain is bound in the P1 subsite, contacting I213, M289, V291, and I300. The thiazole and isopropyl-thiazole groups of ritonavir do not have any stabilizing interactions with the active site. The electron density for these groups is correspondingly poorly defined, and the B-factors, which reflect the precision of the atomic positions, for these parts of the molecule are extremely high. The structure of the pepsin saquinavir complex (FIG. 8B) is similar in that the side chain of the phenylalaninol residue is interacting with the P1′ subsite, but the two ends of the molecule, the quinoline and decahydroisoquinoline moieties, also have poor density and high B-factors. The amprenavir (FIG. 8C) and darunavir (FIG. 8D) structures follow the same pattern. The phenylalaninol residues of both inhibitors occupy the P1′ site, interacting with I213, M289, V291, and I300. The isobutyl groups, mimicking leucine residues, occupy the P1 site, interacting with F111, F117, and I120. In both amprenavir and darunavir, one of the oxygen atoms of the sulfonamide moiety makes a hydrogen bond with the backbone amide of T77. The aniline groups make no polar contacts with the active site. At the opposite end of the molecules where the two compounds differ, the tetrahydrofuran group of amprenavir forms a hydrogen bond with the phenolic oxygen of Y189. The bis-tetrahydrofuran group of darunavir, however, cannot have this interaction with the active site and is limited to van der Waals contacts with I73, T74, I128, and Y189. The structures and binding poses of amprenavir and darunavir were similar and provided no explanation for their disparity in IC₅₀.

TABLE 2
Crystallographic data collection and model refinement statistics

| | Pepsin•Amprenavir | Pepsin•Ritonavir | Pepsin•Darunavir | Pepsin•Saquinavir |
|---|---|---|---|---|
| PDB Entry | 6XCT | 6XCY | 6XD2 | 6XCZ |

Data collection

| Resolution (Å) (last | 72.02-1.99 | (2.04-1.99) | 53.17-2.05 | (2.11-2.05) | 49.34-1.90 | (1.97-1.90) | 57.50-1.89 | (1.93-1.89) |
|---|---|---|---|---|---|---|---|---|
| shell)^{a} | | | | | | | | |
| Space group | P 65 2 2 | | P 65 2 2 | | P 65 2 2 | | P 65 2 2 | |
| a, b, c (Å) | 66.1, 66.1 288.1 | | 66.2, 66.2, 285.5 | | 66.2, 66.2, 290.0 | | 66.4, 66.4, 284.6 | |
| α, β, γ (°) | 90, 90, 120 | | 90, 90, 120 | | 90, 90, 120 | | 90, 90, 120 | |
| R_{merge}^{a} | 0.057 | (0.099) | 0.10 | (0.56) | 0.088 | (0.25) | 0.091 | (0.73) |
| Rₘₑₐₛ^{a} | 0.060 | (0.11) | 0.13 | (0.73) | 0.092 | (0.26) | 0.110 | (0.85) |
| Rₚᵢₘ^{a} | 0.021 | (0.038) | 0.084 | (0.46) | 0.026 | (0.077) | 0.054 | (0.42) |
| CC_{1/2}^{a} | 0.999 | (0.991) | 0.989 | (0.593) | 0.995 | (0.981) | 0.995 | (0.649) |
| No. of unique | 26876 | (1807) | 23751 | (1831) | 30916 | (2998) | 30114 | (1885) |
| reflections^{a} | | | | | | | | |
| Completeness (%)^{a} | 99.9 | (99.5) | 98.2 | (99.8) | 99.85 | (99.90) | 97.8 | (98.0) |
| Multiplicity^{a} | 13.4 | (12.8) | 3.1 | (3.2) | 12.0 | (11.8) | 5.5 | (5.9) |
| [symbol] I/σ(I) [symbol] ^{a} | 31.6 | (18.8) | 6.3 | (2.3) | 35.71 | (10.34) | 7.9 | (1.6) |

Model Refinement

| Reflections used in | 26760 | (2580) | 23747 | (2360) | 30888 | (2995) | 30072 | (2948) |
|---|---|---|---|---|---|---|---|---|
| refinement^{a} | | | | | | | | |
| Reflections used for | 1312 | (114) | 1214 | (116) | 1574 | (125) | 1544 | (145) |
| R_{free}^{a} | | | | | | | | |
| R_{cryst} (R_{free})^{a} | 0.1907 | (0.1906) | 0.2173 | (0.2586) | 0.1997 | (0.1887) | 0.2260 | (0.2941) |

| | | | | |
|---|---|---|---|---|
| Wilson B-factor | 17.73 | 34.87 | 18.85 | 27.03 |
| (Å²) | | | | |
| Average B factor | 20.66 | 45.32 | 22.66 | 38.38 |
| (Å²) | | | | |
| Protein atoms | 19.62 | 44.98 | 21.61 | 37.97 |
| Ligand atoms | 24.29 | 71.10 | 26.63 | 59.65 |
| Solvent | 27.46 | 40.81 | 29.22 | 38.11 |

Root-mean-square (RMS) deviations

| | | | | |
|---|---|---|---|---|
| Bond lengths (Å) | 0.009 | 0.011 | 0.013 | 0.015 |
| Bond angles (°) | 0.77 | 1.48 | 1.02 | 1.68 |
| Coordinate error | 0.14 | 0.12 | 0.15 | 0.10 |
| (Å)^{b} | | | | |

Ramachandran statistics

| | | | | |
|---|---|---|---|---|
| Favored/allowed/outliers | 99.37/0.32/0.32 | 97.82/1.87/0.31 | 99.37/0.32/0.32 | 98.13/1.56/0.31 |
| (%) | | | | |
| Rotamer outliers (%) | 0.00 | 1.82 | 0.00 | 2.92 |
| Clashscore | 0.85 | 2.72 | 2.33 | 2.93 |

^{a}Values in parentheses apply to the high-resolution shell indicated in the resolution row
^{b}Maximum-likelihood based estimates of coordinate error

### In Vivo Mouse Model

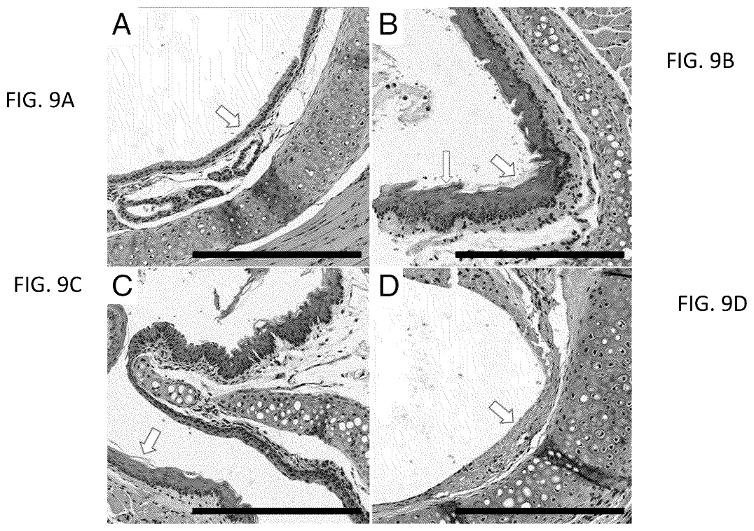

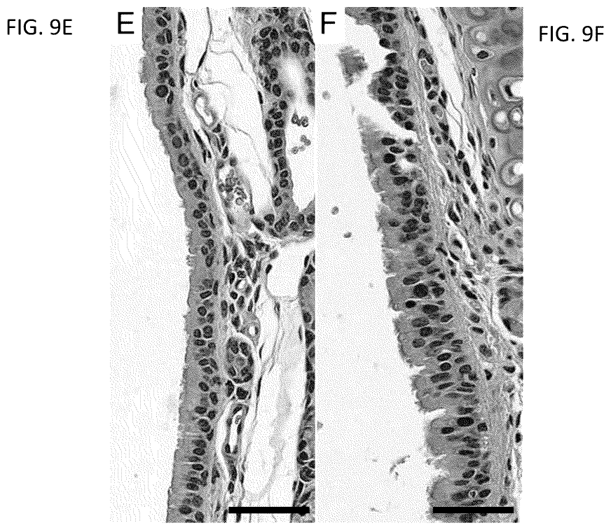

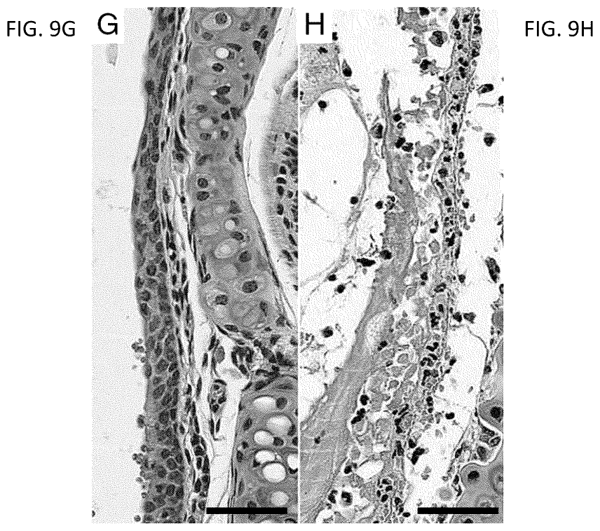

Pepsin-mediated laryngeal epithelial damage was observed at pH 4 and 7 in the mouse in vivo model which employed pepsin with or without acid exposure following mechanical injury of the larynx (FIGS. 9A-9H). Animals in the pH7 control group had normal laryngeal epithelium of 1-2 cells thick with cilia present and no inflammation, keratinization, or necrosis; findings indicated no detectable mucosal damage in the control group due to mechanical injury during the first two weeks of treatment or pH7 solvent. Laryngeal epithelium in the pH4 group was reactive, thickened (3-4 cells thick), and keratinized with loss of cilia. That from the pepsin-pH7 group had an intermediate thickness (2-3 cells), evidence of keratinization, increased nuclear to cytoplasmic ratio and loss of polarization. That from the pepsin-pH4 group exhibited total loss of epithelium due to necrosis and inflammatory cell infiltrate.

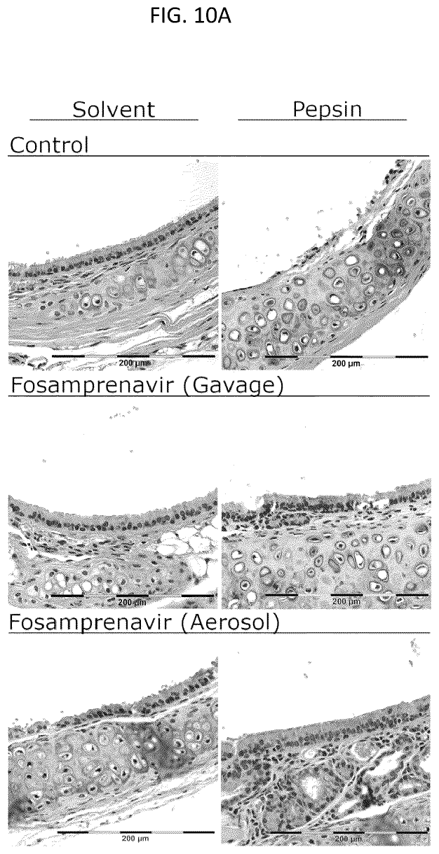

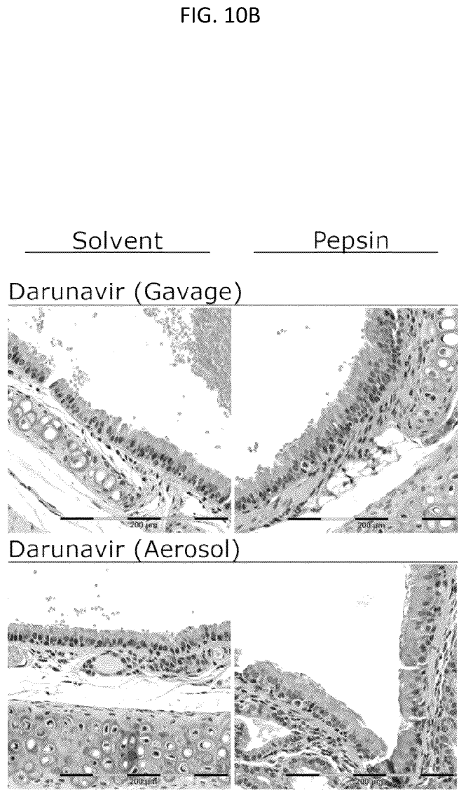

Fosamprenavir gavage equivalent to the dose used to treat HIV in humans prevented pepsin-mediated laryngeal damage, defined as reactive epithelia, increased intraepithelial inflammatory cells, and apoptosis (FIGS. 10A and 10B). Mild reactivity elicited by oral darunavir (absent in darunavir aerosol group; FIGS. 9A-9H) obscured the ability to detect its effect on pepsin-mediated damage. Fosamprenavir aerosol prevented pepsin-mediated laryngeal injury (FIGS. 9A-9H). Darunavir aerosol provided moderate protection against pepsin-mediated damage: while epithelial injury was present (mildly increased intraepithelial inflammatory cells and reactive epithelial cells), no apoptosis was observed as it was in mice treated with pepsin-pH7 and sham inhalation.

### DISCUSSION

For the past two decades, the treatment of LPR has focused on suppressing gastric acid production. With the introduction of MII-pH technology, it is now understood that LPR is commonly nonacidic and that nonacid proximal events are associated with laryngeal endoscopic signs and symptoms^{39-46,48-50,102}. These findings sparked investigations into the nonacidic components of gastric refluxate.

Although bile induces mucosal damage at weakly and non-acid pH experimentally, it has been argued that “there is no evidence that the same mechanism occurs in the human larynx”⁵⁷. The clinical relevance of experimental findings has been called into question. Unconjugated bile acids, which cause damage at neutral-high pH such as that of the laryngopharynx, are rarely found in gastric refluxate.^{56,69} Further, concentrations of bile salts/acids found to damage the larynx and hypopharynx experimentally are 1000-fold greater than those reported in the airways of patients with LPR, GERD and asthma, or lung disease (0.3-50 mM^{96,103,104} versus 0.8-32 uM¹⁰⁵⁻¹⁰⁹) and result in morphologic changes inconsistent with those of LPR patients such as cell membrane ‘blebbing’ ¹¹⁰.

Pepsin is present in all refluxate⁵⁵. Moreover, it is frequently detected in airway tissue and secretions from LPR patients but absent in MII-pH-confirmed reflux-free subjects, and thus may be predictive of reflux-attributed symptoms and disease^{20,39,46,50,55,59,65,67,68,111,112} Pepsin at 1 mg/ml in the stomach is diluted by saliva as it is refluxed proximally. A range of concentrations have been reported in airways: 2.5 μg/ml in saliva, 61.5 μg/ml in nasal secretions^{113,114} and 360 μg/ml in middle ear fluid¹¹⁵. To model chronic LPR within a limited experimental timeframe, 300 μg/ml was employed herein^{1,77,116,117}. Pepsin-mediated damage and inflammatory changes reported in vitro and in vivo, including the histologic changes herein, are consistent with those observed in LPR patients^{62-64,66,70,118-122}. Compelling evidence from multiple groups highlights a major role for pepsin, independent of gastric acid, in reflux-attributed laryngeal symptoms and findings refractory to PPI therapy.

While pepstatin is a potent pepsin inhibitor, its poor water-solubility and pharmacokinetic properties make it a suboptimal therapeutic candidate. Structural data herein indicated that inhibitor binding to the active cleft of pepsin is predominantly stabilized by van der Waals contacts, making rational design of inhibitors difficult. Testing existing inhibitors of other aspartic proteases was therefore deemed the most efficacious route for identification of a pepsin-targeting therapeutic.

There are currently ten commercially available HIV protease inhibitors.¹²³ Seven were amenable to testing in our in vitro binding and inhibition assays and four (amprenavir, ritonavir, saquinavir and darunavir) bound and inhibited pepsin with IC₅₀ in the low micromolar range, validating our hypothesis that existing therapeutic protease inhibitors may exhibit anti-peptic activity. Two drugs were selected for in vivo study based on anti-peptic activity from in vitro assays, cost and reported side effects. While saquinavir exhibits known side effects and interactions (QT prolongation, heart block, high blood lipids and liver problems) and has high cost, amprenavir, ritonavir, and darunavir have minimal side effects (diarrhea, nausea and vomiting).¹²³ Darunavir is more costly than amprenavir and ritonavir, but had the lowest IC₅₀ for pepsin. Darunavir, with the lowest IC₅₀, and fosamprenavir, a prodrug of amprenavir with improved bioavailability and favorable tolerability were therefore selected for assessment in vivo. Given that proximal reflux is inconsistent in surgical models of GER¹²⁴, we employed a model involving mechanical wounding and pepsin/acid instillation which reliably replicates epithelial alterations similar to that observed in patients with LPR^{1,63,70,118,119,125}. Using this model, the human-equivalent dose of fosamprenavir, but not darunavir, prevented pepsin-mediated laryngeal damage. When administered locally by inhalation, treatment with either compound preserved normal laryngeal histology despite pepsin exposure.

The study herein was intended to investigate whether a pepsin inhibitor may prevent laryngeal damage caused by pepsin exposure in vivo. As with any experimental observation, caution should be exercised when translating in vivo findings from a limited number of animals to the clinical situation. Potential differences between mouse and human respiratory pathobiology should be kept in mind while evaluating the clinical implications of these data. Established methods for in vivo modeling of aerodigestive tract damage attributed to GERD and LPR^{1, 96-99} were utilized herein and demonstrated mucosal damage consistent with the clinical presentation of LPR supporting their use for assessing drug prevention of LPR-attributed injury: at the four-week conclusion of treatment, no mucosal damage was detectable given mechanical injury and neutral solvent, whereas multi-layered, reactive epithelia with apoptosis was observed in the pepsin and acid treatment groups. The mouse epiglottis occupies a transitional zone from stratified squamous epithelium of the vocal fold to ciliated pseudostratified columnar epithelium at the supraglottis and infraglottis. To avoid misinterpreting squamous epithelium of the vocal folds as signs of injury, representative images were collected rostral to vocal folds, exclusively from tissue with visible thyroid to serve as a guide. Additional features of reactive epithelia (darkened nuclei, variable nuclear diameter, and increased nuclear to cytoplasmic ratio, intraepithelial inflammatory cells, and apoptosis) in pepsin-treated groups, absent in control pH7.0 and those receiving fosamprenavir or darunavir, confirmed epithelial reactivity due to pepsin and the efficacy of HIV protease for prevention of pepsin-mediated injury. While these data are qualitative and would be bolstered by less subjective quantitative measures, the evidence herein provides initial proof-of-concept that a pepsin-targeting therapeutic may reduce mucosal damage akin to that observed in LPR patients and supports more in-depth investigation. Research is ongoing in our laboratory to examine fosamprenavir protection against pepsin-mediated changes in laryngeal cell viability and inflammatory and carcinogenic gene and protein expressions. Further research is also warranted to determine whether laryngeal protection by fosamprenavir aerosol in vivo was due to systemic activity or local conversion to amprenavir. The intestine is the primary site of fosamprenavir metabolism. Conversion of fosamprenavir to amprenavir by alkaline phosphatase (ALP), which is required for its transepithelial flux and subsequent metabolism by cytochrome P450 enzymes, has been shown to occur via intestinal ALP at or near the surface of Caco-2 cells.^{128,129} It is possible, however, that inhaled fosamprenavir is converted to amprenavir in the airways by serum ALP, just as similar phosphate ester prodrugs are converted by sera collected from healthy subjects. Inhaled fosamprenavir may also be converted by salivary ALP or that expressed by respiratory mucosa and immune cells recruited to tissue injury. Given that ALP is elevated during inflammation¹³²⁻¹³⁴ and carcinogenesis including that of the larynx to which LPR contributes,^{10,74,135-137} ALP may be elevated in LPR-damaged airways thereby increasing fosamprenavir conversion at the desired site of activity. Drug formulations that prolong retention in the aerodigestive tract could further improve local drug conversion and topical activity. Research is ongoing in our laboratory to examine the efficiency of fosamprenavir conversion by laryngeal epithelium, saliva and sera and a dose-response study is underway in the in vivo mouse model to compare the relative efficacies of inhaled fosamprenavir and amprenavir against pepsin-mediated damage.

While additional experimental data will aid our understanding of laryngeal protection by fosamprenavir, LPR symptom improvement will be the ultimate determinant of a successful medical therapy. A randomized placebo-controlled trial therefore represents the best test of a therapeutic compound. Such a trial of fosamprenavir is feasible given that an oral formulation is FDA-approved and an a priori responder definition of clinically meaningful symptom improvement has been established per FDA guidelines.¹³⁸ Intriguingly, pilot epidemiological data (unpublished) support the therapeutic potential of HIV protease inhibitors for LPR and warrant follow-up: among 2,062 adult HIV patients prescribed an HIV protease inhibitor (Froedtert Memorial Lutheran Hospital, Milwaukee, WI, July 2014-2016; Medical College of Wisconsin Institutional Review Board, 13874) just 0.2% had documented LPR whereas the incidence in the general population is 10-34%.^{139,140} These data lend preliminary support for clinical investigation of fosamprenavir as a novel therapeutic approach for LPR.

### CONCLUSION

Compelling evidence highlights a major role for pepsin (independent of gastric acid) in reflux-attributed laryngeal symptoms and endoscopic findings refractory to PPI therapy. Fosamprenavir and darunavir, FDA-approved retroviral therapies for HIV/AIDS, bind and inhibit pepsin, abrogating pepsin-mediated laryngeal inflammation and mucosal damage in an LPR mouse model. These drugs target a foreign virus so are ideal to repurpose, allowing a clinical trial to assess efficacy for a much-needed medical treatment for patients faster than could be achieved with novel compounds. Reformulation for local inhaled delivery could further improve outcomes and limit side effects.

### Data Availability

Structural data are available in the Worldwide Protein Databank (accession codes 6XCY, 6XCT, 6XCZ, 6XD2; http://www.wwpdb.org/).

### Sequences

SEQ ID NO:1 Synthetic peptide substrate for pepsin Lys-Pro-Ala-Glu-Phe-PNP-Arg-Leu (PNP=paranitrophenylalanine)

### REFERENCES

1. Koufman J A. The otolaryngologic manifestations of gastroesophageal reflux disease (GERD): a clinical investigation of 225 patients using ambulatory 24-hour pH monitoring and an experimental investigation of the role of acid and pepsin in the development of laryngeal injury. Laryngoscope 1991; 101:1-78.
2. Vaezi M F. Extraesophageal manifestations of gastroesophageal reflux disease. Clin Cornerstone 2003; 5:32-38; discussion 39-40.
3. Ford C N. Evaluation and management of laryngopharyngeal reflux. JAMA 2005; 294:1534-1540.
4. Bianchi E T, Guerreiro Cardoso P F, Minamoto H, et al. Impact of fundoplication for gastroesophageal reflux in the outcome of benign tracheal stenosis. J Thorac Cardiovasc Surg 2019; 158:1698-1706.
5. Esposito C, Saxena A, Irtan S, Till H, Escolino M. Laparoscopic Nissen Fundoplication: An Excellent Treatment of GERD-Related Respiratory Symptoms in Children-Results of a Multicentric Study. J Laparoendosc Adv Surg Tech A 2018; 28:1023-1028.
6. Gabriel C E, Jones D G. The importance of chronic laryngitis. J Laryngol Otol 1960; 74:349-357.
7. Garg D, Mody M, Pal C, et al. Follicular Bronchiolitis: Two Cases with Varying Clinical and Radiological Presentation. Case Rep Pulmonol 2020; 2020:4564587.
8. Johnston N, Yan J C, Hoekzema C R, et al. Pepsin promotes proliferation of laryngeal and pharyngeal epithelial cells. Laryngoscope 2012; 122:1317-1325.
9. Kelly E A, Samuels T L, Johnston N. Chronic pepsin exposure promotes anchorage-independent growth and migration of a hypopharyngeal squamous cell line. Otolaryngol Head Neck Surg 2014; 150:618-624.
10. Kim S Y, Park B, Lim H, Kim M, Kong I G, Choi H G. Increased risk of larynx cancer in patients with gastroesophageal reflux disease from a national sample cohort. Clin Otolaryngol 2019; 44:534-540.
11. Parsel S M, Wu E L, Riley C A, McCoul E D. Gastroesophageal and Laryngopharyngeal Reflux Associated With Laryngeal Malignancy: A Systematic Review and Meta-analysis. Clin Gastroenterol Hepatol 2019; 17:1253-1264 e1255.
12. Riley C A, Marino M J, Hsieh M C, Wu E L, Wu X C, McCoul E D. Detection of laryngeal carcinoma in the U. S. elderly population with gastroesophageal reflux disease. Head Neck 2019; 41:1434-1440.
13. Tae K, Jin B J, Ji Y B, Jeong J H, Cho S H, Lee S H. The role of laryngopharyngeal reflux as a risk factor in laryngeal cancer: a preliminary report. Clin Exp Otorhinolaryngol 2011; 4:101-104.
14. Wight R, Paleri V, Arullendran P. Current theories for the development of nonsmoking and nondrinking laryngeal carcinoma. Curr Opin Otolaryngol Head Neck Surg 2003; 11:73-77.
15. Altman K W, Stephens R M, Lyttle C S, Weiss K B. Changing impact of gastroesophageal reflux in medical and otolaryngology practice. Laryngoscope 2005; 115:1145-1153.
16. Koufman J A, Amin M R, Panetti M. Prevalence of reflux in 113 consecutive patients with laryngeal and voice disorders. Otolaryngol Head Neck Surg 2000; 123:385-388.
17. Reulbach T R, Belafsky P C, Blalock P D, Koufman J A, Postma G N. Occult laryngeal pathology in a community-based cohort. Otolaryngol Head Neck Surg 2001; 124:448-450.
18. Francis D O, Rymer J A, Slaughter J C, et al. High economic burden of caring for patients with suspected extraesophageal reflux. Am J Gastroenterol 2013; 108:905-911.
19. Gelardi M, Ciprandi G. Focus on gastroesophageal reflux (GER) and laryngopharyngeal reflux (LPR): new pragmatic insights in clinical practice. J Biol Regul Homeost Agents 2018; 32:41-47.
20. Bardhan K D, Strugala V, Dettmar P W. Reflux revisited: advancing the role of pepsin. Int J Otolaryngol 2012; 2012:646901.
21. Martinucci I, de Bortoli N, Savarino E, et al. Optimal treatment of laryngopharyngeal reflux disease. Ther Adv Chronic Dis 2013; 4:287-301.
22. Liu C, Wang H, Liu K. Meta-analysis of the efficacy of proton pump inhibitors for the symptoms of laryngopharyngeal reflux. Braz J Med Biol Res 2016; 49.
23. Reimer C, Bytzer P. Management of laryngopharyngeal reflux with proton pump inhibitors. Ther Cin Risk Manag 2008; 4:225-233.
24. Koufman J A. Laryngopharyngeal reflux is different from classic gastroesophageal reflux disease. Ear Nose Throat J 2002; 81:7-9.
25. Park W, Hicks D M, Khandwala F, et al. Laryngopharyngeal reflux: prospective cohort study evaluating optimal dose of proton-pump inhibitor therapy and pretherapy predictors of response. Laryngoscope 2005; 115:1230-1238.
26. Eherer A J, Habermann W, Hammer H F, Kiesler K, Friedrich G, Krejs G J. Effect of pantoprazole on the course of reflux-associated laryngitis: a placebo-controlled double-blind crossover study. Scand J Gastroenterol 2003; 38:462-467.
27. El-Serag H B, Lee P, Buchner A, Inadomi J M, Gavin M, McCarthy D M. Lansoprazole treatment of patients with chronic idiopathic laryngitis: a placebo-controlled trial. Am J Gastroenterol 2001; 96:979-983.
28. Noordzij J P, Khidr A, Evans B A, et al. Evaluation of omeprazole in the treatment of reflux laryngitis: a prospective, placebo-controlled, randomized, double-blind study. Laryngoscope 2001; 111:2147-2151.
29. Steward D L, Wilson K M, Kelly D H, et al. Proton pump inhibitor therapy for chronic laryngo-pharyngitis: a randomized placebo-control trial. Otolaryngol Head Neck Surg 2004; 131:342-350.
30. Vaezi M F, Richter J E, Stasney C R, et al. Treatment of chronic posterior laryngitis with esomeprazole. Laryngoscope 2006; 116:254-260.
31. Wo J M, Koopman J, Harrell S P, Parker K, Winstead W, Lentsch E. Double-blind, placebo-controlled trial with single-dose pantoprazole for laryngopharyngeal reflux. Am J Gastroenterol 2006; 101:1972-1978; quiz 2169.
32. Lam P K, Ng M L, Cheung T K, et al. Rabeprazole is effective in treating laryngopharyngeal reflux in a randomized placebo-controlled trial. Clin Gastroenterol Hepatol 2010; 8:770-776.
33. Reichel O, Dressel H, Wiederanders K, Issing W J. Double-blind, placebo-controlled trial with esomeprazole for symptoms and signs associated with laryngopharyngeal reflux. Otolaryngol Head Neck Surg 2008; 139:414-420.
34. Vaezi M E. Gastroesophageal reflux-related chronic laryngitis: con. Arch Otolaryngol Head Neck Surg 2010; 136:908-909.
35. Lien H C, Wang C C, Liang W M, et al. Composite pH predicts esomeprazole response in laryngopharyngeal reflux without typical reflux syndrome. Laryngoscope 2013; 123:1483-1489.
36. Masaany M, Marina M B, Sharifa Ezat W P, Sani A. Empirical treatment with pantoprazole as a diagnostic tool for symptomatic adult laryngopharyngeal reflux. J Laryngol Otol 2011; 125:502-508.
37. Kahrilas P J. When proton pump inhibitors fail. Clin Gastroenterol Hepatol 2008; 6:482-483.
38. Barry D W, Vaezi M F. Laryngopharyngeal reflux: More questions than answers. Cleve Clin J Med 2010; 77:327-334.
39. Lechien J R, Bock J M, Carroll T L, Akst L M. Is empirical treatment a reasonable strategy for laryngopharyngeal reflux? A contemporary review. Clin Otolaryngol 2020.
40. Sharma N, Castell D O. Further comment on proton pump inhibitor failures. Clin Gastroenterol Hepatol 2009; 7:363.
41. Tamhankar A P, Peters J H, Portale G, et al. Omeprazole does not reduce gastroesophageal reflux: new insights using multichannel intraluminal impedance technology. J Gastrointest Surg 2004; 8:890-897; discussion 897-898.
42. Tutuian R, Mainie I, Agrawal A, Adams D, Castell D O. Nonacid reflux in patients with chronic cough on acid-suppressive therapy. Chest 2006; 130:386-391.
43. Tutuian R, Vela M F, Hill E G, Mainie I, Agrawal A, Castell D O. Characteristics of symptomatic reflux episodes on Acid suppressive therapy. Am J Gastroenterol 2008; 103:1090-1096.
44. Falk G L, Van der Wall H, Burton L, Falk M G, O'Donnell H, Vivian S J. Fundoplication for laryngopharyngeal reflux despite preoperative dysphagia. Ann R Coll Surg Engl 2017; 99:224-227.
45. Iqbal M, Batch A J, Spychal R T, Cooper B T. Outcome of surgical fundoplication for extraesophageal (atypical) manifestations of gastroesophageal reflux disease in adults: a systematic review. J Laparoendosc Adv Surg Tech A 2008; 18:789-796.
46. Klimara M J, Randall D R, Allen J, Figueredo E, Johnston N. Proximal reflux: biochemical mediators, markers, therapeutic targets, and clinical correlations. Ann N Y Acad Sci 2020; 1481:127-138.
47. Lechien J R, Dapri G, Dequanter D, et al. Surgical Treatment for Laryngopharyngeal Reflux Disease: A Systematic Review. JAMA Otolaryngol Head Neck Surg 2019; 145:655-666.
48. Mainie I, Tutuian R, Shay S, et al. Acid and non-acid reflux in patients with persistent symptoms despite acid suppressive therapy: a multicentre study using combined ambulatory impedance-pH monitoring. Gut 2006; 55:1398-1402.
49. Sidwa F, Moore A L, Alligood E, Fisichella P M. Surgical Treatment of Extraesophageal Manifestations of Gastroesophageal Reflux Disease. World J Surg 2017; 41:2566-2571.
50. Zhang C, Hu Z W, Yan C, et al. Nissen fundoplication vs proton pump inhibitors for laryngopharyngeal reflux based on pH-monitoring and symptom-scale. World J Gastroenterol 2017; 23:3546-3555.
51. Giacchi R J, Sullivan D, Rothstein S G. Compliance with anti-reflux therapy in patients with otolaryngologic manifestations of gastroesophageal reflux disease. Laryngoscope 2000; 110:19-22.
52. McGlashan J A, Johnstone L M, Sykes J, Strugala V, Dettmar P W. The value of a liquid alginate suspension (Gaviscon Advance) in the management of laryngopharyngeal reflux. Eur Arch Otorhinolaryngol 2009; 266:243-251.
53. Zalvan C H, Hu S, Greenberg B, Geliebter J. A Comparison of Alkaline Water and Mediterranean Diet vs Proton Pump Inhibition for Treatment of Laryngopharyngeal Reflux. JAMA Otolaryngol Head Neck Surg 2017; 143:1023-1029.
54. Koufman J A. Low-acid diet for recalcitrant laryngopharyngeal reflux: therapeutic benefits and their implications. Ann Otol Rhinol Laryngol 2011; 120:281-287.
55. Samuels T L, Johnston N. Pepsin as a marker of extraesophageal reflux. Ann Otol Rhinol Laryngol 2010; 119:203-208.
56. Ali M S, Parikh S, Chater P, Pearson J P. Bile acids in laryngopharyngeal refluxate: will they enhance or attenuate the action of pepsin? Laryngoscope 2013; 123:434-439.
57. Campagnolo A M, Priston J, Thoen R H, Medeiros T, Assuncao A R. Laryngopharyngeal reflux: diagnosis, treatment, and latest research. Int Arch Otorhinolaryngol 2014; 18:184-191.
58. Johnston N, Dettmar P W, Bishwokarma B, Lively M O, Koufman J A. Activity/stability of human pepsin: implications for reflux attributed laryngeal disease. Laryngoscope 2007; 117:1036-1039.
59. Samuels T L, Johnston N. Pepsin as a causal agent of inflammation during nonacidic reflux. Otolaryngol Head Neck Surg 2009; 141:559-563.
60. Tan J J, Wang L, Mo T T, Wang J, Wang M G, Li X P. Pepsin promotes IL-8 signaling-induced epithelial-mesenchymal transition in laryngeal carcinoma. Cancer Cell Int 2019; 19:64.
61. Piper D W, Fenton B H. pH stability and activity curves of pepsin with special reference to their clinical importance. Gut 1965; 6:506-508.
62. Axford S E, Sharp N, Ross P E, et al. Cell biology of laryngeal epithelial defenses in health and disease: preliminary studies. Ann Otol Rhinol Laryngol 2001; 110:1099-1108.
63. Gill G A, Johnston N, Buda A, et al. Laryngeal epithelial defenses against laryngopharyngeal reflux: investigations of E-cadherin, carbonic anhydrase isoenzyme III, and pepsin. Ann Otol Rhinol Laryngol 2005; 114:913-921.
64. Johnston N, Bulmer D, Gill G A, et al. Cell biology of laryngeal epithelial defenses in health and disease: further studies. Ann Otol Rhinol Laryngol 2003; 112:481-491.
65. Johnston N, Dettmar P W, Ondrey F G, Nanchal R, Lee S H, Bock J M. Pepsin: biomarker, mediator, and therapeutic target for reflux and aspiration. Ann N Y Acad Sci 2018; 1434:282-289.
66. Johnston N, Knight J, Dettmar P W, Lively M O, Koufman J. Pepsin and carbonic anhydrase isoenzyme III as diagnostic markers for laryngopharyngeal reflux disease. Laryngoscope 2004; 114:2129-2134.
67. Johnston N, Wells C W, Samuels T L, Blumin J H. Pepsin in nonacidic refluxate can damage hypopharyngeal epithelial cells. Ann Otol Rhinol Laryngol 2009; 118:677-685.
68. Johnston N, Wells C W, Samuels T L, Blumin J H. Rationale for targeting pepsin in the treatment of reflux disease. Ann Otol Rhinol Laryngol 2010; 119:547-558.
69. Pearson J P, Parikh S, Orlando R C, et al. Review article: reflux and its consequences—the laryngeal, pulmonary and oesophageal manifestations. Conference held in conjunction with the 9th International Symposium on Human Pepsin (ISHP) Kingston-upon-Hull, U K, 21-23 Apr. 2010. Aliment Pharmacol Ther 2011; 33 Suppl 1:1-71.
70. Rees L E, Pazmany L, Gutowska-Owsiak D, et al. The mucosal immune response to laryngopharyngeal reflux. Am J Respir Crit Care Med 2008; 177:1187-1193.
71. Samuels T L, Altman K W, Gould J C, et al. Esophageal pepsin and proton pump synthesis in barrett's esophagus and esophageal adenocarcinoma. Laryngoscope 2019; 129:2687-2695.
72. Sasaki C T, Toman J, Vageli D. The In Vitro Effect of Acidic-Pepsin on Nuclear Factor KappaB Activation and Its Related Oncogenic Effect on Normal Human Hypopharyngeal Cells. PLoS One 2016; 11:e0168269.
73. Samuels T L, Johnston N. Pepsin in gastroesophageal and extraesophageal reflux: molecular pathophysiology and diagnostic utility. Curr Opin Otolaryngol Head Neck Surg 2020; 28:401-409.
74. Samuels T L, Zimmermann M T, Zeighami A, et al. RNA Sequencing Reveals Cancer-Associated Changes in Laryngeal Cells Exposed to Non-Acid Pepsin. Laryngoscope 2021; 131:121-129.
75. Hurley B P, Jugo R H, Snow R F, et al. Pepsin Triggers Neutrophil Migration Across Acid Damaged Lung Epithelium. Sci Rep 2019; 9:13778.
76. Kim J H, Jang S J, Yun J W, Jung M H, Woo S H. Effects of pepsin and pepstatin on reflux tonsil hypertrophy in vitro. PLoS One 2018; 13:e0207090.
77. Nagahama K, Yamato M, Nishio H, Takeuchi K. Essential role of pepsin in pathogenesis of acid reflux esophagitis in rats. Dig Dis Sci 2006; 51:303-309.
78. Samuels T L, Pearson A C, Wells C W, Stoner G D, Johnston N. Curcumin and anthocyanin inhibit pepsin-mediated cell damage and carcinogenic changes in airway epithelial cells. Ann Otol Rhinol Laryngol 2013; 122:632-641.
79. Niu K, Guo C, Teng S, et al. Pepsin promotes laryngopharyngeal neoplasia by modulating signaling pathways to induce cell proliferation. PLoS One 2020; 15:e0227408.
80. Lea W A, Simeonov A. Fluorescence polarization assays in small molecule screening. Expert Opin Drug Discov 2011; 6:17-32.
81. Roberts N B, Taylor W H. Comparative pepstatin inhibition studies on individual human pepsins and pepsinogens 1,3 and 5(gastricsin) and pig pepsin A. J Enzyme Inhib Med Chem 2003; 18:209-217.
82. Jolley M E. Fluorescence Polarization Assays for the Detection of Proteases and Their Inhibitors. J Biomol Screen 1996; 1:33-38.
83. Olp M D, Kalous K S, Smith B C. ICEKAT: an interactive online tool for calculating initial rates from continuous enzyme kinetic traces. BMC Bioinformatics 2020; 21:186.
84. Luft J R, DeTitta G T. A method to produce microseed stock for use in the crystallization of biological macromolecules. Acta Crystallogr D Biol Crystallogr 1999; 55:988-993.
85. Battye T G, Kontogiannis L, Johnson O, Powell H R, Leslie A G. iMOSFLM: a new graphical interface for diffraction-image processing with MOSFLM. Acta Crystallogr D Biol Crystallogr 2011; 67:271-281.
86. Otwinowski Z, Minor W. Processing of X-ray diffraction data collected in oscillation mode. Methods Enzymol 1997; 276:307-326.
87. McCoy A J, Grosse-Kunstleve R W, Adams P D, Winn M D, Storoni L C, Read R J. Phaser crystallographic software. J Appl Crystallogr 2007; 40:658-674.
88. Adams P D, Afonine P V, Bunkoczi G, et al. PHENIX: a comprehensive Python-based system for macromolecular structure solution. Acta Crystallogr D Biol Crystallogr 2010; 66:213-221.
89. Afonine P V, Mustyakimov M, Grosse-Kunstleve R W, Moriarty N W, Langan P, Adams P D. Joint X-ray and neutron refinement with phenix.refine. Acta Crystallogr D Biol Crystallogr 2010; 66:1153-1163.
90. Emsley P, Cowtan K. Coot: model-building tools for molecular graphics. Acta Crystallogr D Biol Crystallogr 2004; 60:2126-2132.
91. Emsley P, Lohkamp B, Scott W G, Cowtan K. Features and development of Coot. Acta Crystallogr D Biol Crystallogr 2010; 66:486-501.
92. Long F, Nicholls R A, Emsley P, et al. AceDRG: a stereochemical description generator for ligands. Acta Crystallogr D Struct Biol 2017; 73:112-122.
93. Chen V B, Arendall W B, 3rd, Headd J J, et al. MolProbity: all-atom structure validation for macromolecular crystallography. Acta Crystallogr D Biol Crystallogr 2010; 66:12-21.
94. Joosten R P, Long F, Murshudov G N, Perrakis A. The PDB_REDO server for macromolecular structure model optimization. IUCrJ 2014; 1:213-220.
95. Caicedo-Granados E, Galbraith A R, Schachern M G, et al. N-methylnitrosourea-induced carcinoma as a model for laryngeal carcinogenesis. Head Neck 2014; 36:1802-1806.
96. Adhami T, Goldblum J R, Richter J E, Vaezi M F. The role of gastric and duodenal agents in laryngeal injury: an experimental canine model. Am J Gastroenterol 2004; 99:2098-2106.
97. Little F B, Koufman J A, Kohut R I, Marshall R B. Effect of gastric acid on the pathogenesis of subglottic stenosis. Ann Otol Rhinol Laryngol 1985; 94:516-519.
98. Roh J L, Yoon Y H. Effect of acid and pepsin on glottic wound healing: a simulated reflux model. Arch Otolaryngol Head Neck Surg 2006; 132:995-1000.
99. Yellon R F, Szeremeta W, Grandis J R, Diguisseppe P, Dickman P S. Subglottic injury, gastric juice, corticosteroids, and peptide growth factors in a porcine model. Laryngoscope 1998; 108:854-862.
100. Xie Y, Longest P W, Xu Y H, Wang J P, Wiedmann T S. In vitro and in vivo lung deposition of coated magnetic aerosol particles. J Pharm Sci 2010; 99:4658-4668.
101. Fujinaga M, Chernaia M M, Tarasova N I, Mosimann S C, James M N. Crystal structure of human pepsin and its complex with pepstatin. Protein Sci 1995; 4:960-972.
102. Lechien J R, Akst L M, Hamdan A L, et al. Evaluation and Management of Laryngopharyngeal Reflux Disease: State of the Art Review. Otolaryngol Head Neck Surg 2019; 160:762-782.
103. Sasaki C T, Doukas S G, Doukas P G, Vageli D P. Weakly Acidic Bile Is a Risk Factor for Hypopharyngeal Carcinogenesis Evidenced by DNA Damage, Antiapoptotic Function, and Premalignant Dysplastic Lesions In Vivo. Cancers (Basel) 2021; 13.
104. Figueiredo A A, Sales T, Nicolau L A D, et al. Laryngeal Mucosa Alterations in Mice Model of Gastroesophageal Reflux: Effects of Topical Protection. Laryngoscope 2020; 130:E889-e895.
105. Blondeau K, Mertens V, Vanaudenaerde B A, et al. Gastro-oesophageal reflux and gastric aspiration in lung transplant patients with or without chronic rejection. Eur Respir J 2008; 31:707-713.
106. De Corso E, Baroni S, Salonna G, et al. Impact of bile acids on the severity of laryngopharyngeal reflux. Clin Otolaryngol 2021; 46:189-195.
107. D'Ovidio F, Mura M, Tsang M, et al. Bile acid aspiration and the development of bronchiolitis obliterans after lung transplantation. J Thorac Cardiovasc Surg 2005; 129:1144-1152.
108. McQuaid K R, Laine L, Fennerty M B, Souza R, Spechler S J. Systematic review: the role of bile acids in the pathogenesis of gastro-oesophageal reflux disease and related neoplasia. Aliment Pharmacol Ther 2011; 34:146-165.
109. Perng D W, Chang K T, Su K C, et al. Exposure of airway epithelium to bile acids associated with gastroesophageal reflux symptoms: a relation to transforming growth factor-beta1 production and fibroblast proliferation. Chest 2007; 132:1548-1556.
110. Hopwood D, Bateson M C, Milne G, Bouchier I A. Effects of bile acids and hydrogen ion on the fine structure of oesophageal epithelium. Gut 1981; 22:306-311.
111. Calvo-Henriquez C, Ruano-Ravina A, Vaamonde P, Martinez-Capoccioni G, Martin-Martin C. Is Pepsin a Reliable Marker of Laryngopharyngeal Reflux? A Systematic Review. Otolaryngol Head Neck Surg 2017; 157:385-391.
112. Weitzendorfer M, Antoniou S A, Schredl P, et al. Pepsin and oropharyngeal pH monitoring to diagnose patients with laryngopharyngeal reflux. Laryngoscope 2019.
113. Klimara M J, Johnston N, Samuels T L, et al. Correlation of salivary and nasal lavage pepsin with MII-pH testing. Laryngoscope 2020; 130:961-966.
114. Klimara M J, Samuels T L, Johnston N, Chun R H, McCormick M E. Detection of Pepsin in Oral Secretions of Infants with and without Laryngomalacia. Ann Otol Rhinol Laryngol 2020; 129:224-229.
115. Sone M, Yamamuro Y, Hayashi H, Niwa Y, Nakashima T. Otitis media in adults as a symptom of gastroesophageal reflux. Otolaryngol Head Neck Surg 2007; 136:19-22.
116. Durkes A, Sivasankar M P. In vivo investigation of acidified pepsin exposure to porcine vocal fold epithelia. Laryngoscope 2016; 126:E12-17.
117. Erickson E, Sivasankar M. Simulated reflux decreases vocal fold epithelial barrier resistance. Laryngoscope 2010; 120:1569-1575.
118. Amin S M, Abdel Maged K H, Naser A Y, Aly B H. Laryngopharyngeal reflux with sore throat: an ultrastructural study of oropharyngeal epithelium. Ann Otol Rhinol Laryngol 2009; 118:362-367.
119. Andrews T M, Orobello N. Histologic versus pH probe results in pediatric laryngopharyngeal reflux. Int J Pediatr Otorhinolaryngol 2013; 77:813-816.
120. Lechien J R, Schindler A, Robotti C, Lejeune L, Finck C. Laryngopharyngeal reflux disease in singers: Pathophysiology, clinical findings and perspectives of a new patient-reported outcome instrument. Eur Ann Otorhinolaryngol Head Neck Dis 2019; 136:S39-S43.
121. Lipan M J, Reidenberg J S, Laitman J T. Anatomy of reflux: a growing health problem affecting structures of the head and neck. Anat Rec B New Anat 2006; 289:261-270.
122. Powell J, Cocks H C. Mucosal changes in laryngopharyngeal reflux—prevalence, sensitivity, specificity and assessment. Laryngoscope 2013; 123:985-991.
123. Lv Z, Chu Y, Wang Y. HIV protease inhibitors: a review of molecular selectivity and toxicity. HIV AIDS (Auckl) 2015; 7:95-104.
124. Pham T H, Genta R M, Spechler S J, Souza R F, Wang D H. Development and characterization of a surgical mouse model of reflux esophagitis and Barrett's esophagus. J Gastrointest Surg 2014; 18:234-240; discussion 240-231.
125. Gaynor E B. Gastroesophageal reflux as an etiologic factor in laryngeal complications of intubation. Laryngoscope 1988; 98:972-979.
126. Perkins E L, Basu S, Garcia G J M, Buckmire R A, Shah R N, Kimbell J S. Ideal Particle Sizes for Inhaled Steroids Targeting Vocal Granulomas: Preliminary Study Using Computational Fluid Dynamics. Otolaryngol Head Neck Surg 2018; 158:511-519.
127. Lungova V, Verheyden J M, Herriges J, Sun X, Thibeault S L. Ontogeny of the mouse vocal fold epithelium. Dev Biol 2015; 399:263-282.
128. Wire M B, Shelton M J, Studenberg S. Fosamprenavir: clinical pharmacokinetics and drug interactions of the amprenavir prodrug. Clin Pharmacokinet 2006; 45:137-168.
129. Furfine E S, Baker C T, Hale M R, et al. Preclinical pharmacology and pharmacokinetics of GW433908, a water-soluble prodrug of the human immunodeficiency virus protease inhibitor amprenavir. Antimicrob Agents Chemother 2004; 48:791-798.
130. Dasgupta A, Schlette E. Rapid in vitro conversion of fosphenytoin into phenytoin in sera of patients with liver disease: role of alkaline phosphatase. J Clin Lab Anal 2001; 15:244-250.
131. Bourne G H. Alkaline phosphatase in taste buds and nasal mucosa. Nature 1948; 161:445.
132. Rader B A. Alkaline Phosphatase, an Unconventional Immune Protein. Front Immunol 2017; 8:897.
133. Li H, Zhao Y, Li W, Yang J, Wu H. Critical role of neutrophil alkaline phosphatase in the antimicrobial function of neutrophils. Life Sci 2016; 157:152-157.
134. Reale M F, M.; Grilli, A.; Barbacane, R. C.; Placido, F.; Porreca, E.; Conti, P. Induction of alkaline phosphatase generation by il-1β and LPS on human neutrophils and macrophages and lack of inhibition by interleukin-1 receptor antagonist. Inflammopharmacology 1995; 3:25-34.
135. Chen L, Zeng H, Yang J, et al. Survival and prognostic analysis of preoperative inflammatory markers in patients undergoing surgical resection for laryngeal squamous cell carcinoma. BMC Cancer 2018; 18:816.
136. Hammond K D, Mohamed E, Gregor R T. Alkaline phosphatase and phosphoamino acid phosphatases in normal and cancerous tissues of the human larynx. Biochem Med Metab Biol 1990; 43:75-79.
137. Sharma U, Pal D, Prasad R. Alkaline phosphatase: an overview. Indian J Clin Biochem 2014; 29:269-278.
138. Lien H C, Wang C C, Lee S W, et al. Responder Definition of a Patient-Reported Outcome Instrument for Laryngopharyngeal Reflux Based on the U S FDA Guidance. Value Health 2015; 18:396-403.
139. Kamani T, Penney S, Mitra I, Pothula V. The prevalence of laryngopharyngeal reflux in the English population. Eur Arch Otorhinolaryngol 2012; 269:2219-2225.
140. Lowden M, McGlashan J A, Steel A, Strugala V, Dettmar P W. Prevalence of symptoms suggestive of extra-oesophageal reflux in a general practice population in the U K. Logoped Phoniatr Vocol 2009; 34:32-35.
141. Nikki Johnston, PhD; Tina L. Samuels, MS; Christopher J. Goetz, BS; Leggy A. Arnold, PhD; Brian C. Smith, PhD; Donna Seabloom, BS; Beverly Wuertz, BS; Frank Ondrey, MD, PhD; Timothy S. Wiedmann, PhD; Nemanja Vuksanovic, PhD; Nicholas R. Silvaggi, PhD; Alexander C. MacKinnon, MD, PhD; James Miller, MD, MPH; Jonathan Bock, MD; Joel H. Blumin, MD. Oral and Inhaled Fosamprenavir Reverses Pepsin-Induced Damaged in a Laryngopharyngeal Reflux Mouse Model. Laryngoscope, 00:1-11, 2022.
142. Tina L. Samuels M S; Simon Blaine-Sauer B S; Ke Yan PhD; Nikki Johnston PhD. Amprenavir inhibits pepsin-mediated laryngeal epithelial disruption and E-cadherin cleavage in vitro. Laryngoscope Investigative Otolaryngology, 2023; 1-10.
143. Simon Blaine-Sauer; Tina L. Samuels; Ke Yan; and Nikki Johnston. The Protease Inhibitor Amprenavir Protects against Pepsin-Induced Esophageal Epithelial Barrier Disruption and Cancer-Associated Changes. Int. J. Mol. Sci. 2023, 24, 6765.
144. Alexandra Lesnick BS, Tina L. Samuels MS, Donna Seabloom BS, Beverly Wuertz BS, Abhilash Ojha MS, Davis Seelig D V M PhD DACVPY, Frank Ondrey MD, Timothy S. Wiedmann PhD, Chris Hogan PhD, Emma Torii BVSc MANZCVS DACVP, Hui Ouyang PhD, Ke Yan PhD, Guilherme J. M. Garcia PhD, Jonathan M. Bock MD FACS, Nikki Johnston PhD. Title: Inhaled fosamprenavir for laryngopharyngeal reflux: Toxicology and fluid dynamics modeling; Running title: Inhaled fosamprenavir for LPR. July 2023 Draft Manuscript for The Laryngoscope journal.

### Additional Aspects

The following disclosure can be described in accordance with the following numbered clauses.
Clause 1. An oral sustained release formulation for treating reflux comprising: an effective amount of an HIV protease inhibitor; sodium alginate and a pharmaceutically acceptable carrier.
Clause 2. The oral sustained release formulation of clause 1, wherein the HIV protease inhibitor and sodium alginate form improve sustained release over at least 30 minutes.
Clause 3. The oral formulation of clause 1 or 2, wherein the HIV protease inhibitor is amprenavir, darunavir, ritonavir, saquinavir, or any combination thereof.
Clause 4. The oral formulation of any one of the preceding the clauses, wherein the HIV protease inhibitor is amprenavir or its prodrug fosamprenavir.
Clause 5. The oral formulation of any one of the preceding clauses, the formulation comprising one or more of a suspending agent, a preservative, a sweetener, a flavoring, water, and a combination thereof.
Clause 6. The oral formulation of any one of the preceding clauses, wherein the formulation is a liquid.
Clause 7. A method of treating reflux in a subject in need thereof, the method comprising oral administering the formulation of any one of clauses 1-5 to a subject to treat the reflux.
Clause 8. The method of clause 7, wherein the HIV protease inhibitor is capable of binding to and inhibiting the enzymatic activity of pepsin.
Clause 9. The method of any one of clauses 7-8, wherein the HIV protease inhibitor is administered twice daily at a dosage of about 1.4 g or lower.
Clause 10. The method of any one of clauses 7-9, wherein the subject has an airway refluxcondition selected from laryngopharyngeal reflux (LPR), gastropharyngeal reflux (GPR), and esophagopharyngeal reflux (EPR).
Clause 11. The method of clause 10, wherein the subject's condition is refractory to treatment with a proton pump inhibitor (PPI).
Clause 12. The method of any one of clauses 7-10, wherein the method reduces laryngeal mucosal damage and inflammation.
Clause 13. The method of any one of clauses 7-12, wherein the subject has gastroesophageal reflux disease (GERD), preferably GERD that is refractory to proton pump inhibition.
Clause 14. Use of the composition of any one of clauses 1-5 for the treatment of reflux in a subject in need thereof, wherein the subject has a condition selected from the group consisting of laryngopharyngeal reflux (LPR), gastropharyngeal reflux (GPR), esophagopharyngeal reflux (EPR), or GERD refractory to protein pump inhibition.

What is claimed:

1. An oral sustained release formulation for treating reflux comprising:
an effective amount of an HIV protease inhibitor;
sodium alginate and a pharmaceutically acceptable carrier.

2. The oral sustained release formulation of claim 1, wherein the HIV protease inhibitor and sodium alginate form improve sustained release over at least 30 minutes.

3. The oral formulation of claim 1, wherein the HIV protease inhibitor is amprenavir, darunavir, ritonavir, saquinavir, or any combination thereof.

4. The oral formulation of claim 1, wherein the HIV protease inhibitor is amprenavir or its prodrug fosamprenavir.

5. The oral formulation of claim 1, the formulation comprising one or more of a suspending agent, a preservative, a sweetener, a flavoring, water, and a combination thereof.

6. The oral formulation of claim 1, wherein the formulation is a liquid.

7. A method of treating reflux in a subject in need thereof, the method comprising oral administering the formulation of claim 1 to a subject to treat the reflux.

8. The method of claim 7, wherein the HIV protease inhibitor is capable of binding to and inhibiting the enzymatic activity of pepsin.

9. The method of claim 7, wherein the HIV protease inhibitor is administered twice daily at a dosage of about 1.4 g or lower.

10. The method of claim 7, wherein the subject has an airway refluxcondition selected from laryngopharyngeal reflux (LPR), gastropharyngeal reflux (GPR), and esophagopharyngeal reflux (EPR).

11. The method of claim 10, wherein the subject's condition is refractory to treatment with a proton pump inhibitor (PPI).

12. The method of claim 7, wherein the method reduces laryngeal mucosal damage and inflammation.

13. The method of claim 7, wherein the subject has gastroesophageal reflux disease (GERD), preferably GERD that is refractory to proton pump inhibition.

14. Use of the composition of claim 1 for the treatment of reflux in a subject in need thereof, wherein the subject has a condition selected from the group consisting of laryngopharyngeal reflux (LPR), gastropharyngeal reflux (GPR), esophagopharyngeal reflux (EPR), or GERD refractory to protein pump inhibition.
